(19) United States
(12) Patent Application Publication
(10) Pub. No.: US 2026/0182974 A1
(43) Pub. Date: Jul. 2, 2026

(54) ORAL FLUID COLLECTION DEVICES, KITS, AND METHODS

(71) Applicant: Neogen Corporation, Lansing, MI (US)

(72) Inventors: Randall Bachtel, Lawrenceville, GA (US); John Cain, River Falls, WI (US); James Cargile, Smyrna, GA (US); James Clarke, St. Joseph, MI (US); Erik ` Cook, Lexington, KY (US); Tina German, St. Joseph, MI (US); Daniel Grubb, Versailles, KY (US); Taylor Leigh, Milton, GA (US); Jason Lye, Atlanta, GA (US); Stephen Phelps, Lilburn, GA (US); Steven Schadler, St. Joseph, MI (US); Peter Shipp, Jr., Woodstock, GA (US); Michael Sloan, Atlanta, GA (US); Noah Underwood, Atlanta, GA (US); Lloyd Vasilakes, Hudson, WI (US)

(21) Appl. No.: 18/996,650

(22) PCT Filed: Jul. 18, 2023
(86) PCT No.: PCT/US2023/070406

§ 371 Date: Jan. 17, 2025

Related U.S. Application Data
(60) Provisional application No. 63/390,436, filed on Jul. 19, 2022.
(60) Provisional application No. 63/504,519, filed on May 26, 2023.

(51) Int. Cl. A61B 10/00

(52) U.S. Cl. CPC A61B 10/0051; A61B 10/0096; A61B 2010/0006

(57) ABSTRACT

Devices and methods for collecting oral fluids and determining a sufficiency of the fluid collected. The device includes a handpiece, a sample volume adequacy indicator, and a clamping means for holding a collection pad during collection and ejecting the collection pad after sufficient fluid has been collected in the collection pad.

This application claims priority to U.S. provisional application No. 63/504,519 filed on May 26, 2023, and to U.S. provisional application No. 63/390,436 filed on Jul. 19, 2022, and which are incorporated herein in their entirety by reference.

### FIELD

The present disclosure relates generally to devices and methods for collecting fluids. More specifically, the present disclosure relates to devices and methods for collecting oral fluids and determining a sufficiency of the fluid collected for testing applications.

### SUMMARY

Disclosed embodiments describe devices, kits, and methods for collecting oral fluids and determining a sufficiency of the fluid collected. In some embodiments, an oral fluid collection device comprise a handpiece, a sample volume adequacy indicator (SVAI) integrated into the handpiece and configured to provide a visual indicator of sufficient fluid collection, and means for retaining a collection pad so that the collection pad is detachably coupled to the handpiece during oral fluid collection and ejected from the handpiece after oral fluid collection so that only the collection pad is coupled and decoupled from the handpiece. The means for retaining may comprise a clamp configured to retain a portion of the collection pad within a distal end of the handpiece when the collection pad is coupled to the handpiece.

The device may further comprise an inner shaft, wherein the joint is integrally formed with or coupled to the inner shaft. Longitudinal movement of the inner shaft towards the distal end of the handpiece may eject the collection pad from the handpiece and the clamp.

The clamp may have two opposing arms with substantially smooth surfaces with release ramps and a pad locking component, or with release ramps and pad retaining projections. The clamp may have two opposing arms each having a plurality of perforations or surface texture configured to couple and decouple the collection pad. The clamp may be formed from one perforated continuous film, that folds around one end of the collection pad and is prevented from being ejected along with the pad by attachment to the inner shaft.

The device may have an inner shaft, so that the SVAI may have windows in the handpiece, and a wick is positioned in the inner shaft so that a portion of the wick is visible through the aligned handpiece windows.

The wick may be positioned in contact with the collection pad when the collection pad is coupled to the handpiece. The wick may further have a visual indicator configured to migrate via a wicking action with the application of saliva to the aligned handpiece and inner shaft windows.

One or more of the inner shaft and handpiece may be constructed so to prevent relative rotation of the inner shaft with respect to the handpiece. Inner shaft and handpiece both may have at least one flat surface that prevents mutual rotation of the inner shaft and handpiece. In some embodiments, one or more of the inner shaft and handpiece have a generally square cross section.

In some embodiments, ejection of the pad may be accompanied by one or more of a haptic snap and/or an audible click feedback.

Wick and the collection pad may be formed of materials having either the same or different densities. In some embodiments, wick and the collection pad may be one piece.

Force needed to dislodge the dry pad from the clamp may be in the range of 0.75 Newtons to 10 Newtons when tested in the Dry Pad Retention Strength Protocol. The force needed to dislodge the wet pad from the clamp may be in the range of 0.75 Newtons to about 10 Newtons when tested in the Wet Pad Retention Strength Protocol.

According to some embodiments, an oral fluid collection kit may have an oral fluid collection device having a handpiece, a sample volume adequacy indicator (SVAI) integrated into the handpiece and configured to provide a visual indicator of sufficient fluid collection, and a clamp configured to retain a portion of the collection pad within a distal end of the handpiece when the collection pad is detachably coupled to the handpiece and to eject only the collection pad from the handpiece, The kit may also have a collection container configured to receive the collection pad when ejected from the handpiece, wherein the collection container contains a buffer solution.

The buffer may be a buffer containing a detergent and preservative. In some embodiments, the buffer may be a neutral phosphate buffer. The device in the kit may have an inner shaft, so that the clamp is integrally formed with or coupled to the inner shaft, and longitudinal movement of the inner shaft towards the distal end of the handpiece ejects the collection pad from the handpiece and the clamp, or the clamp is integrally formed with the handpiece and the collection pad is coupled via the clamp, wherein longitudinal movement of the inner shaft towards the distal end of the handpiece ejects the collection pad from the handpiece and the clamp.

The clamp may include two opposing arms each having a plurality of perforations or surface texture configured to couple and decouple the collection pad.

The device in the kit may further have an inner shaft, such that the SVAI comprises windows in the handpiece that are aligned with windows in the inner shaft, and a wick positioned in the inner shaft so that a portion of the wick is visible through the aligned handpiece and inner shaft windows. The wick may be positioned in contact with the collection pad when the collection pad is coupled to the handpiece, and wherein the wick further comprises a visual indicator configured to migrate via a wicking action with the application of saliva to the aligned handpiece and inner shaft windows. The wick and the collection pad are formed of materials having the same or different densities. The wick and the collection pad may be one piece.

In some embodiments, the force needed to dislodge the dry pad from the clamp may be in the range of about 0.75 Newtons to about 10 Newtons when tested in the Dry Pad Retention Strength Protocol. The force needed to dislodge the wet pad from the clamp may be in the range of about 0.75 Newtons to about 10 Newtons when tested in the Wet Pad Retention Strength Protocol.

In accordance with some embodiments, a method of manufacturing an oral fluid collection device may comprise forming handpiece and at least one inner shaft component, and a knob, inserting a wick into the at least one inner shaft component, and inserting the wick with the inner at least one shaft component into the handpiece to form an sample volume adequacy indicator (SVAI); and coupling a collection pad to the at least one inner shaft components or the handpiece so that the collection pad contacts the wick.

The at least one inner shaft component may be formed to include a dual lever joint that is integrally formed with or coupled to the inner shaft, and the collection pad is coupled via the dual lever joint, or a clamp may be integrally formed with the handpiece and the collection pad coupled via the clamp. The method may further comprise depositing a visual indicator in the wick, and coupling the knob to the assembled handpiece and inner shaft components so that the inner shaft components extend into a recess in the knob.

The SVAI may have windows in the handpiece, and the wick positioned in the inner shaft so that a portion of the wick is visible through the aligned handpiece windows. The handpiece and inner shaft components are formed by injection molding.

One or more aspects and embodiments may be incorporated in a different embodiment although not specifically described. That is, all aspects and embodiments may be combined in any way or combination.

### BRIEF DESCRIPTION OF THE DRAWINGS

The drawings disclose exemplary embodiments in which like reference characters designate the same or similar parts throughout the figures of which:

FIG. 1 is a partial exploded view of an oral fluid collection device:

FIG. 2A is perspective view of a fully assembled oral fluid collection device according to the disclosed embodiments;

FIG. 211 is a longitudinal cross-sectional view of the fully assembled oral fluid collection device of FIG. 2A;

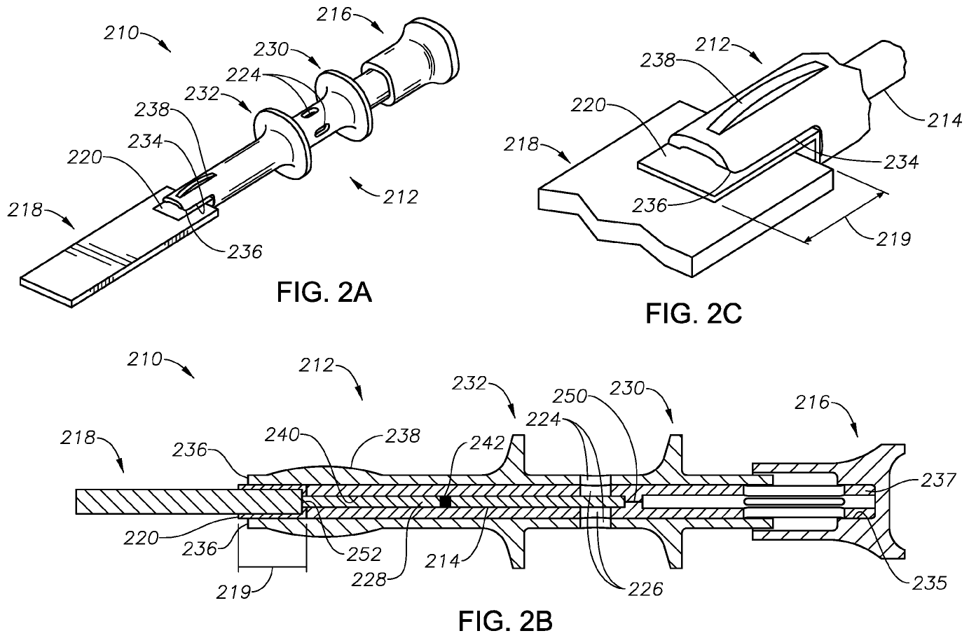

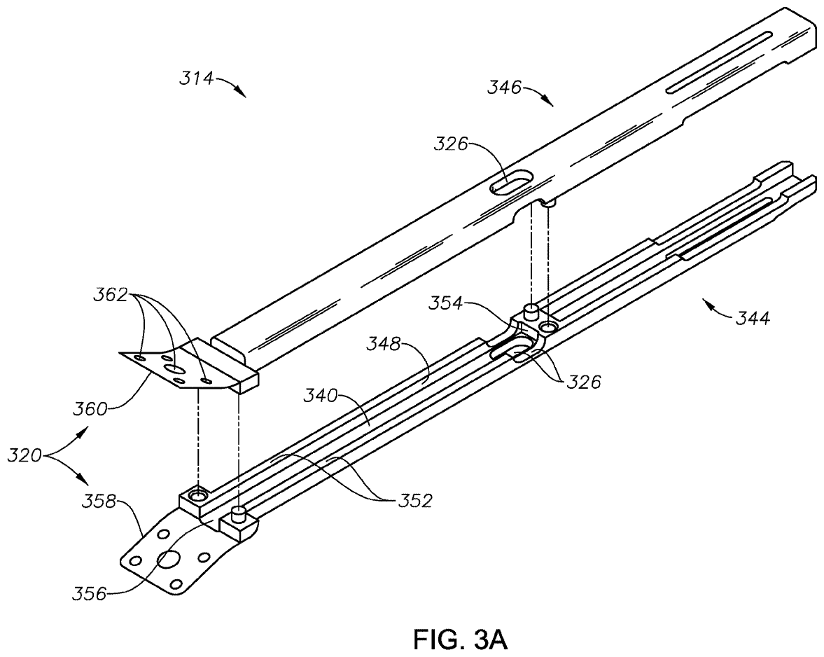

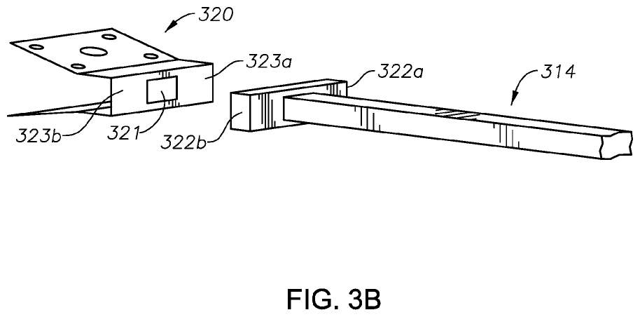

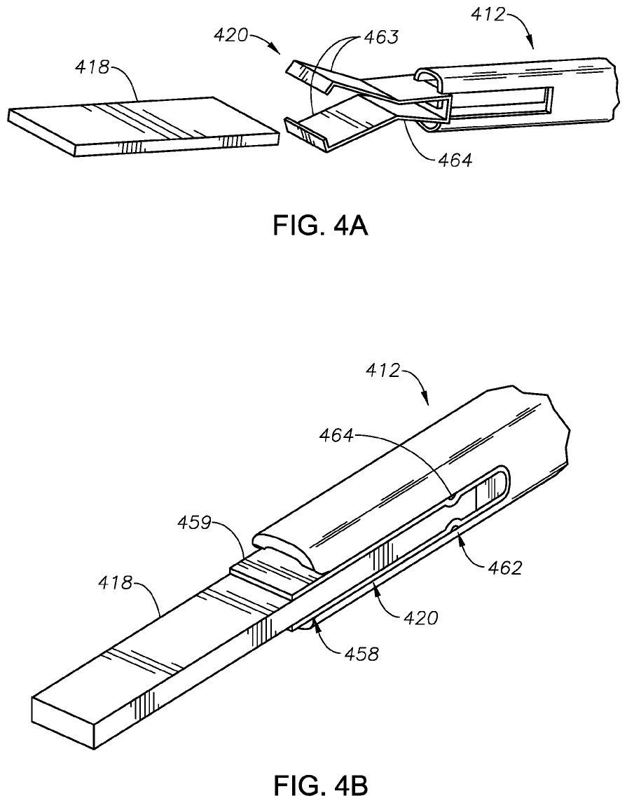

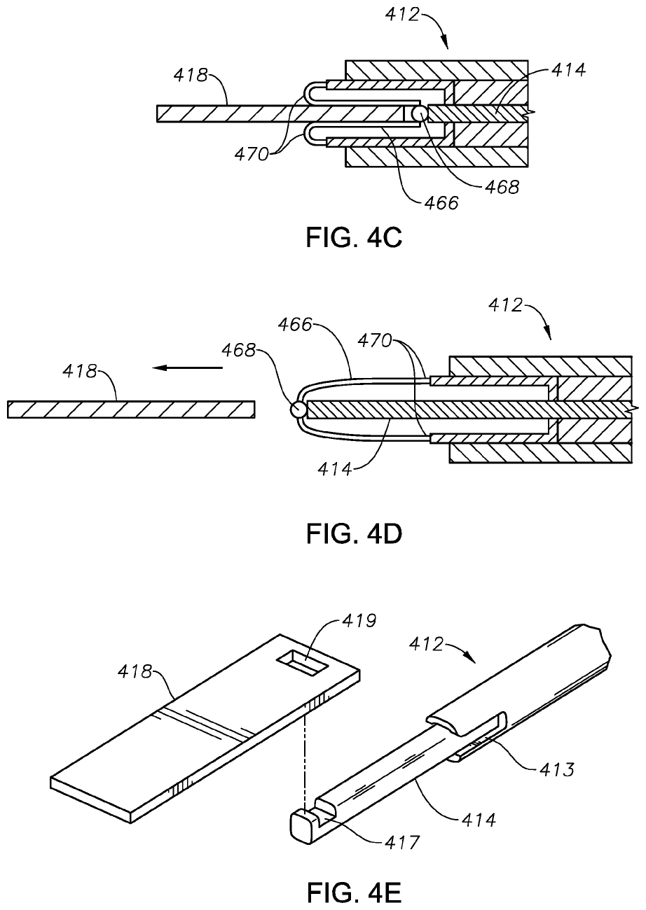

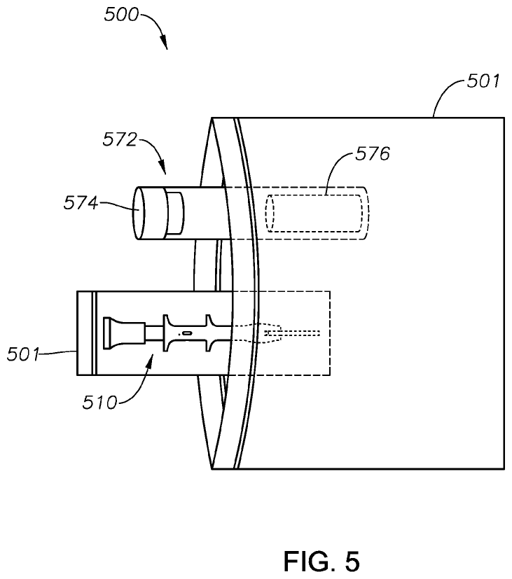

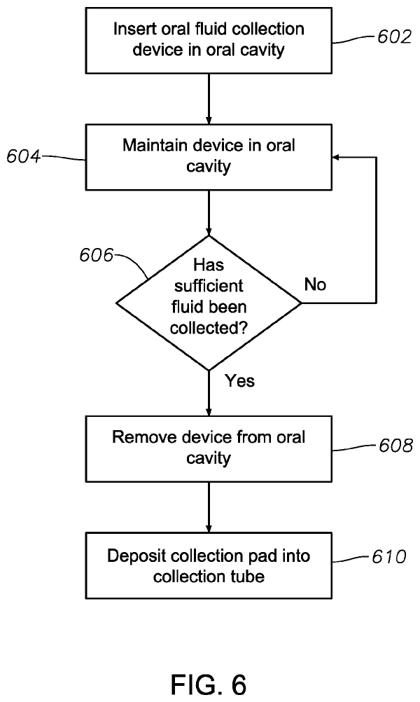

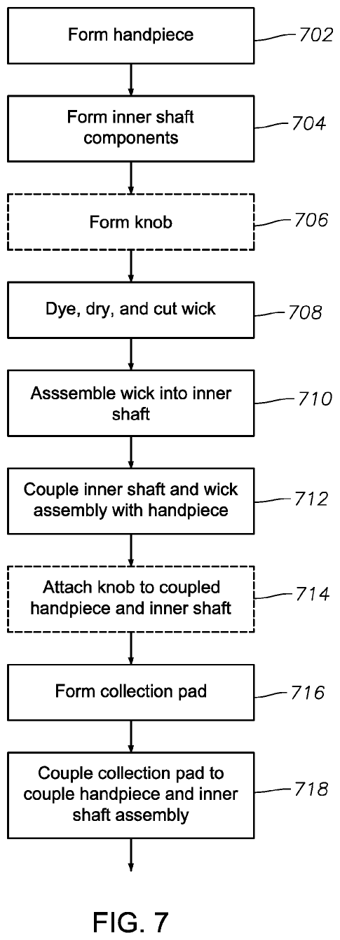

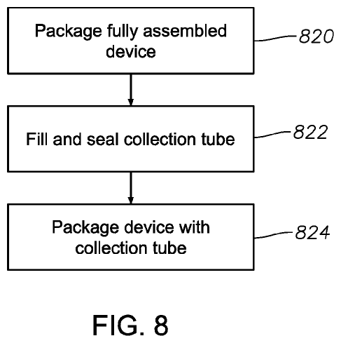

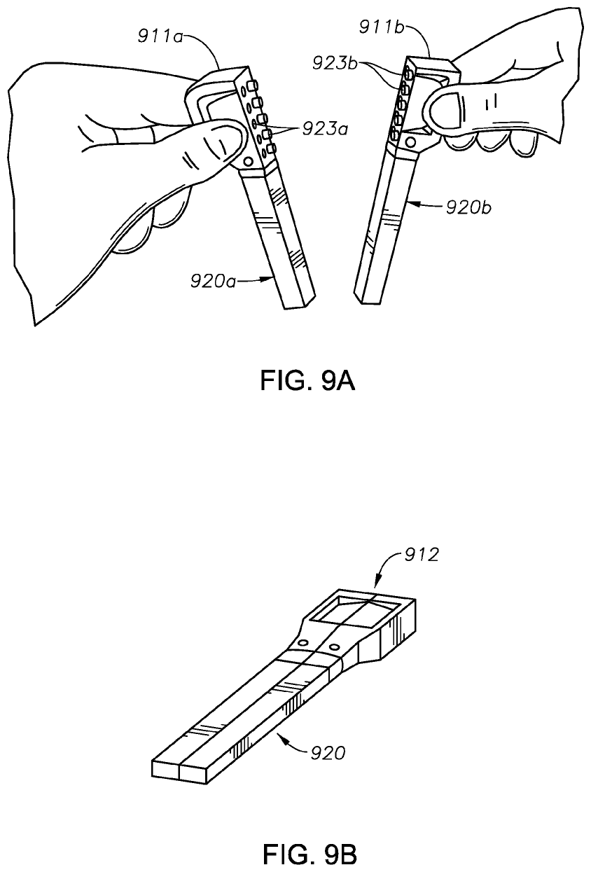

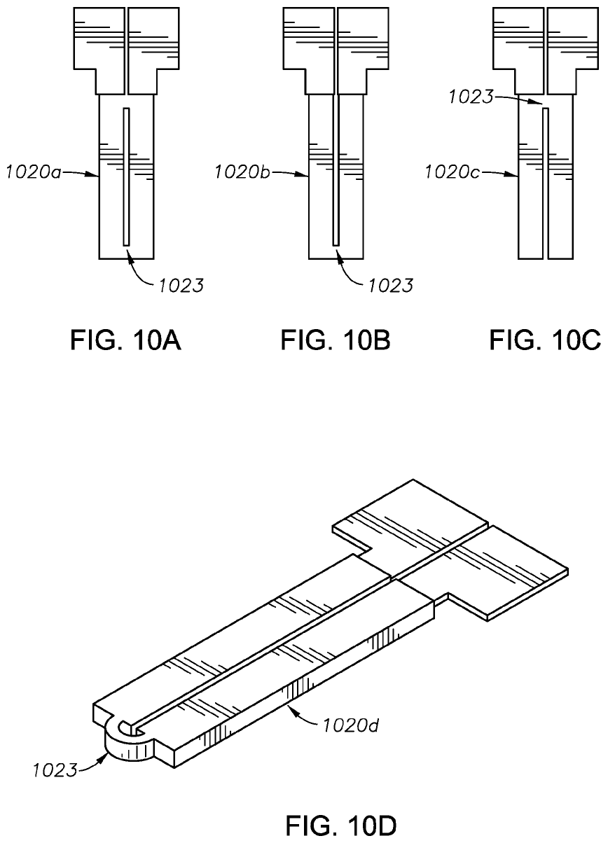

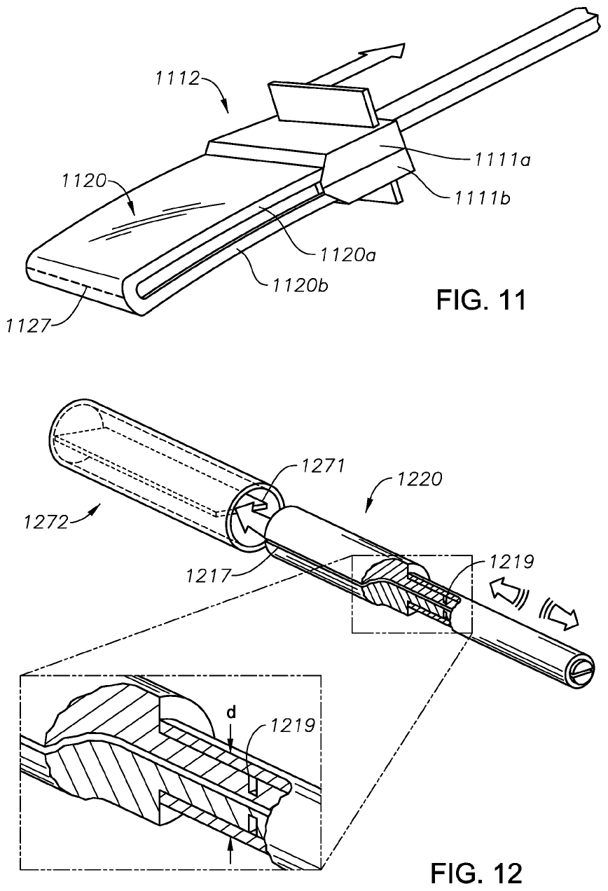

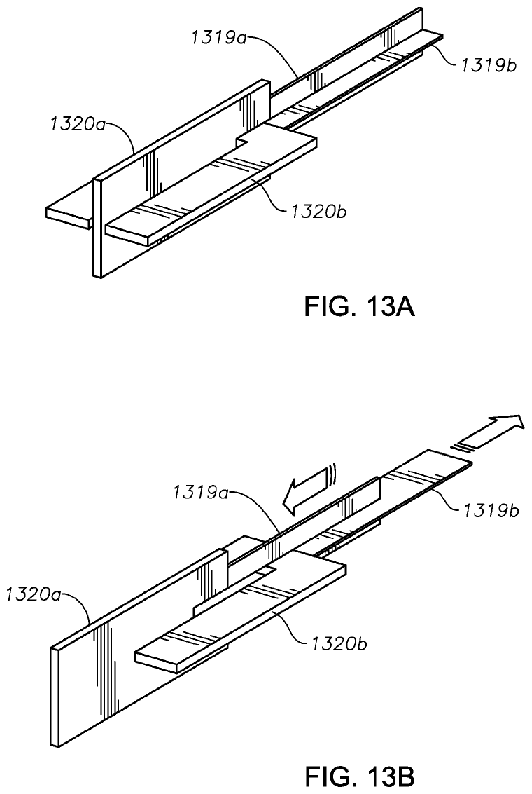

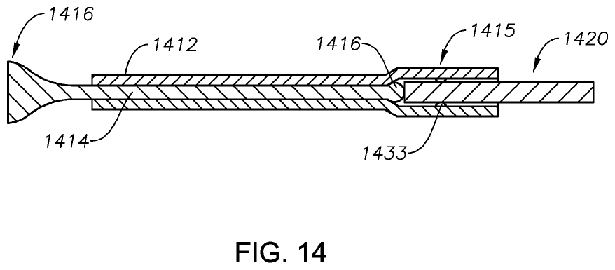

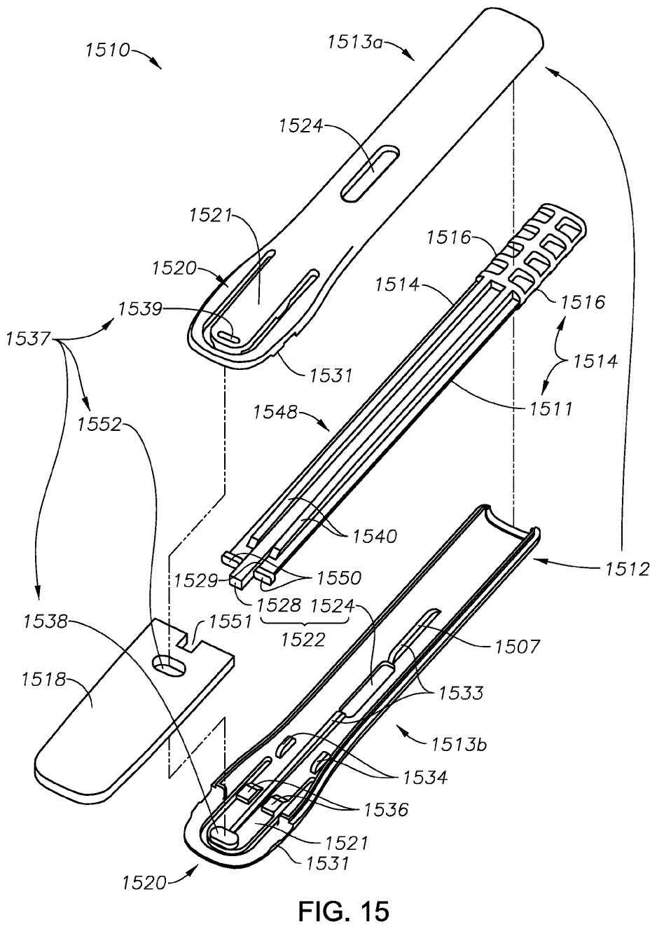

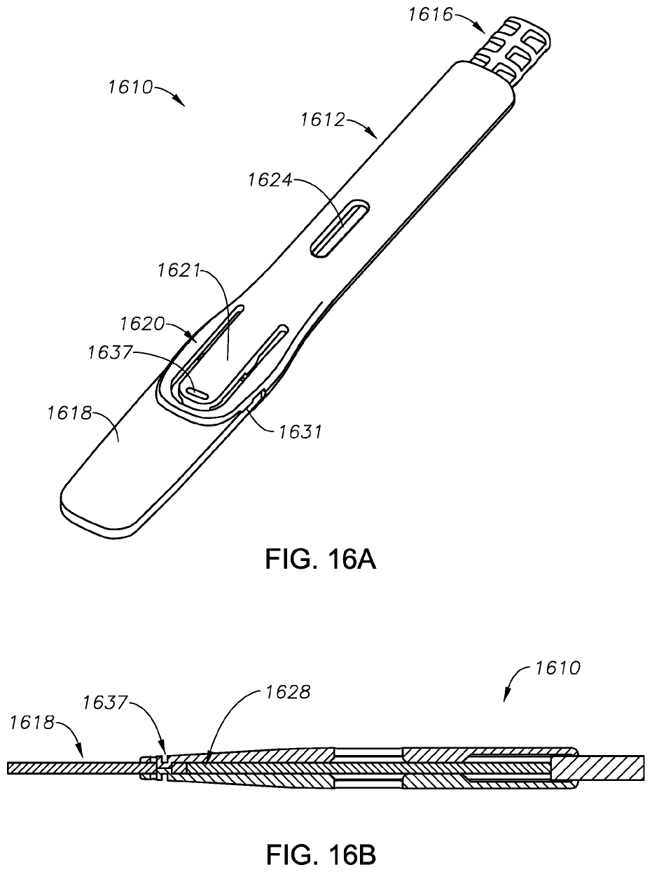

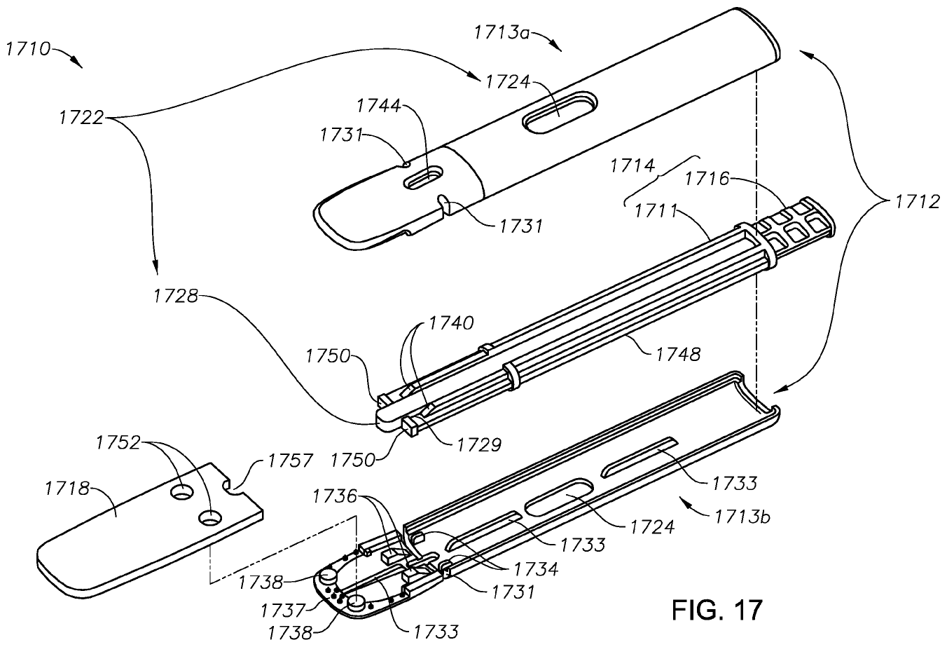

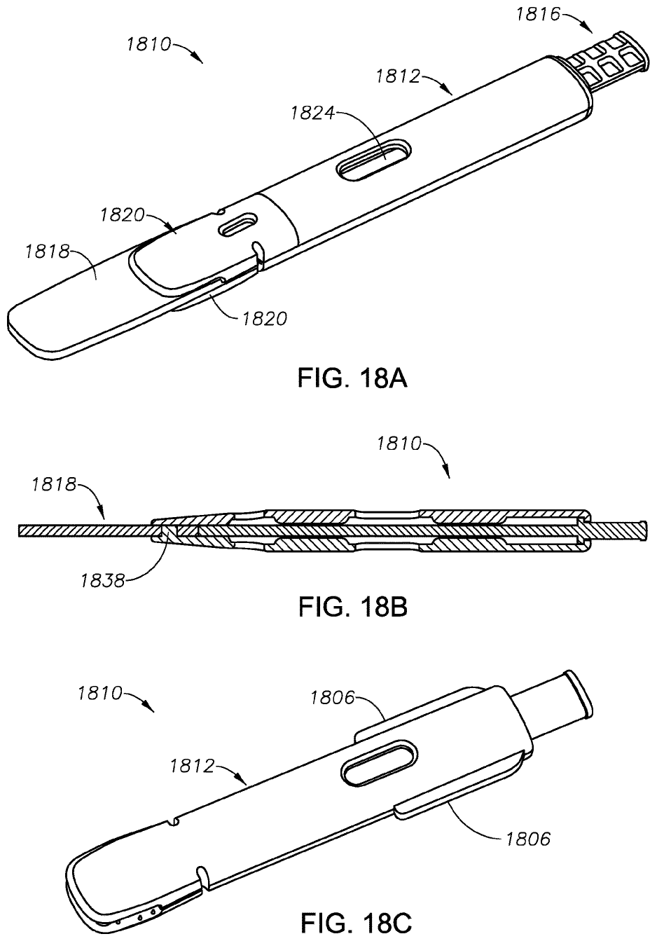

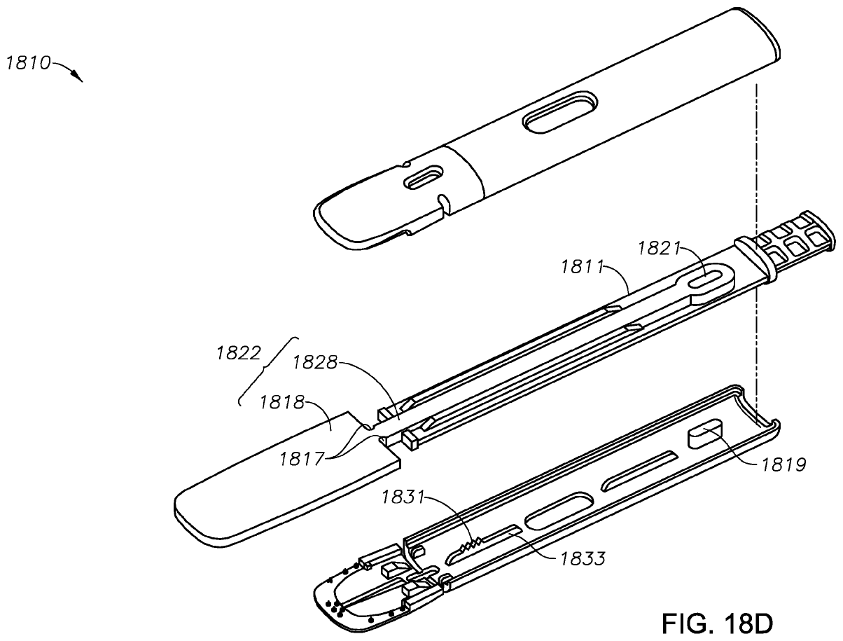

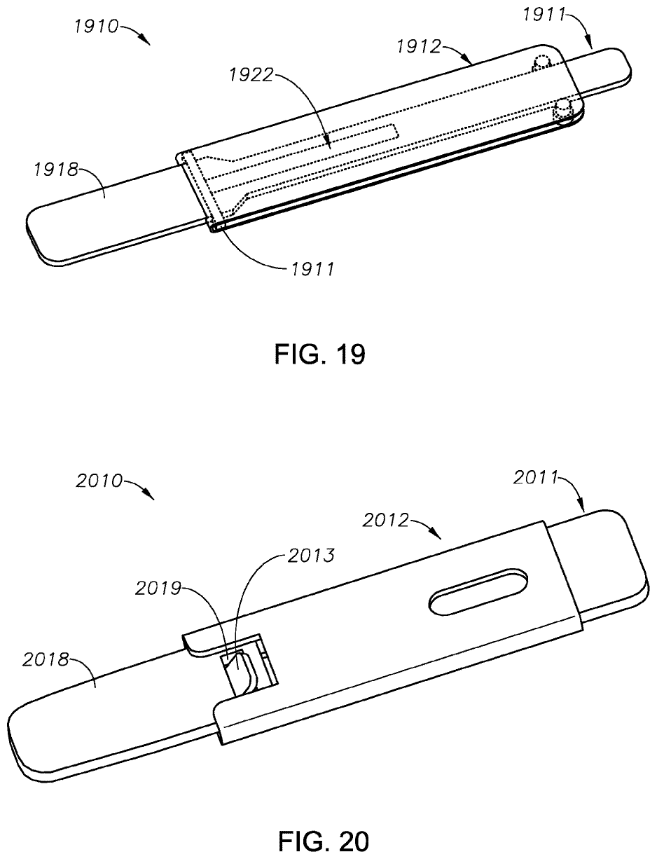

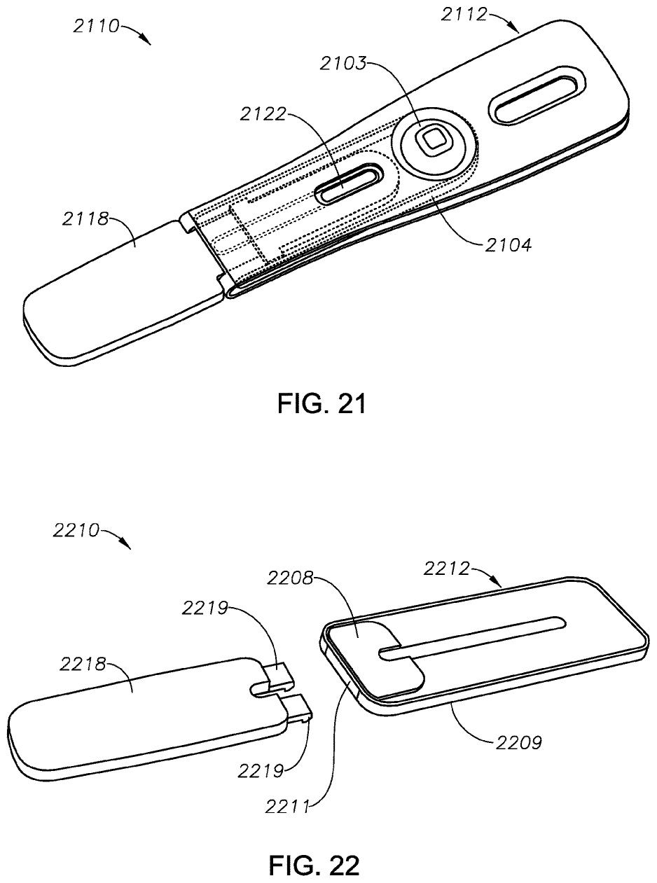

FIG. 2C is an enlarged cross-sectional view of a portion of the fully assembled oral fluid collection device of FIG. 2A;

FIG. 3A is a view of the interior and exterior of first and second body components that form the inner shaft according to the disclosed embodiments;

FIG. 313 is a view of an alternative clamp and inner shaft configuration according to the disclosed embodiments;

FIGS. 4A-E show alternative clamp assemblies in accordance with the disclosed embodiments;

FIG. 5 illustrates a kit for oral fluid collection in accordance with the disclosed embodiments;

FIG. 6 is a flowchart of a method for administering oral fluid collection according to the disclosed embodiments;

FIG. 7 is a flowchart of a method of manufacturing an oral fluid collection device according to the disclosed embodiments;

FIG. 8 is a flowchart of manufacturing an oral fluid collection kit according to the disclosed embodiments;

FIGS. 9A-9B illustrate an oral fluid collection device according to another embodiment;

FIGS. 10A-10D illustrate oral fluid collection device swab configurations according to the disclosed embodiments:

FIG. 11 illustrates an oral fluid collection device according to another embodiment;

FIG. 12 illustrates an oral fluid collection device according to another embodiment;

FIGS. 13A-13B illustrate an oral fluid collection device according to another embodiment;

FIG. 14 illustrates an oral fluid collection device according to another embodiment;

FIG. 15 is a partial exploded view of an oral fluid collection device according to another embodiment;

FIG. 16A is perspective view of a fully assembled oral fluid collection device according to the disclosed embodiments;

FIG. 16B is a longitudinal cross-sectional view of the fully assembled oral fluid collection device of FIG. 16A;

FIG. 17 is a partial exploded view of an oral fluid collection device according to another embodiment;

FIG. 18A is perspective view of a fully assembled oral fluid collection device according to the disclosed embodiments;

FIG. 18B is a longitudinal cross-sectional view of the fully assembled oral fluid collection device of FIG. 18A;

FIG. 18C is a perspective view of a fully assembled oral fluid collection device with an alternative handpiece according to the disclosed embodiments;

FIG. 18D is a partially exploded view of an oral fluid collection device with an alternative unitary wick and collection pad according to the disclosed embodiments;

FIG. 19 illustrates an oral fluid collection device according to another embodiment;

FIG. 20 illustrates an oral fluid collection device according to another embodiment;

FIG. 21 illustrates an oral fluid collection device according to another embodiment; and

FIG. 22 illustrates an oral fluid collection device according to another embodiment.

### DETAILED DESCRIPTION

For the purposes of promoting an understanding of the principles of the disclosure, reference will now be made to a number of illustrative embodiments illustrated in the drawings and specific language will be used to describe the same.

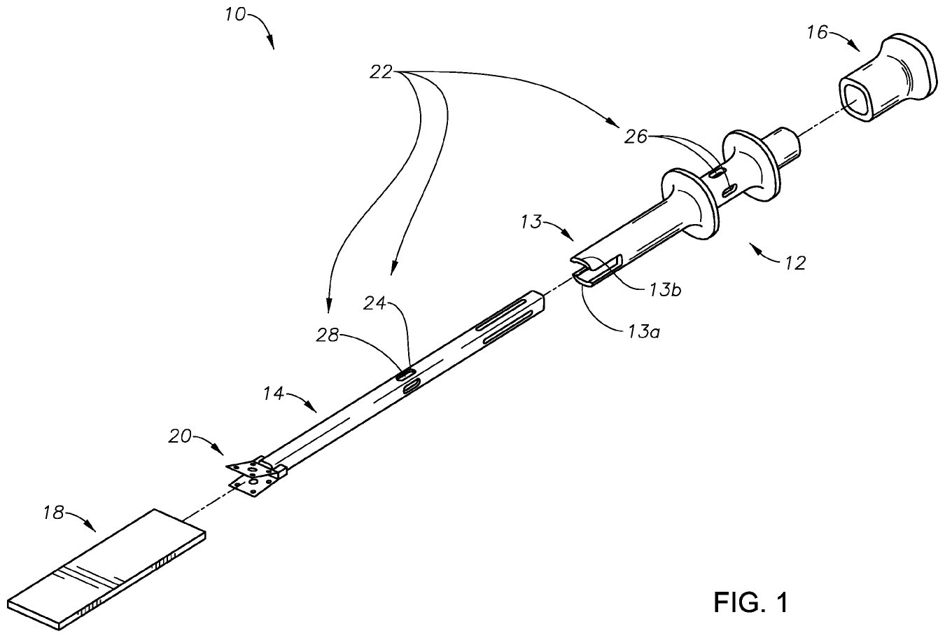

In the illustrative embodiment as shown in FIG. 1, a partial exploded view of the oral fluid collection device 10 having a handpiece 12, an inner shaft, 14, a knob 16, and a sample collection pad 18 is shown. Inner shaft 14 is configured to be inserted or otherwise connected to handpiece 12. In one embodiment, a distal end of handpiece 12 is configured with a socket 13, into which the proximal end of inner shaft 14 may be secured. As illustrated, socket 13 may be concave for reception of a connection with a matingly configured proximal end of inner shaft 14. Retention means, such as clamp 20 holding the pad in place during shipping and fluid collection may have two main components: projections (13a and 13b) at the distal end of handpiece 12 exert a gripping force perpendicular to the plane of the sample collection pad 18. The pad 18 is lined on either side with clamps 20, preferably formed of polymeric materials with perforations that may enhance the grip exerted by the two projections (13a and 13b). These dual lever clamps 20 may collectively be referred to as a “duck bill” configuration, because of the resemblance to a beak. The overall configuration enhances the grip on the pad 18, as well as participates in the ejection or release of the pad 18 at the end of the collection process. Knob 16 is formed to fit over proximal end of handpiece 12 in an assembled position, as described further in FIGS. 2A-B. Inner shaft 14 has a clamp 20 at its distal end, in particular a dual lever clamp 20 which may hold the sample collection pad 18 in the assembled position during shipment and fluid collection, and release the sample collection pad 18 from the handpiece 12 and inner shaft 14 after fluid collection is completed.

A sample volume adequacy indicator (SVAI) 22 may include one or more sample volume adequacy indicator windows 24, 26, that align on each of the handpiece 12 and inner shaft 14, respectively, as well as a SVAI wick 28 that may signal a user visibly through the one or more SVAI windows 24, 26 when a sufficient volume of oral fluid has been collected.

Referring to FIG. 2A, an exemplary embodiment of an assembled oral fluid collection device 210 is shown. The dual lever clamps 220 are shown assembled and holding a sample collection pad 218. The handpiece 212 may include two radially extending finger guides such as an ejector stop 230, and a mouth stop 232, with the SVAI windows 224 positioned between the ejector stop 230 and the mouth stop 232. Ejector stop 230 cooperates with movement of the knob 216 towards the ejector stop 230 during motion to eject the sample collection pad 218, allowing for single-handed operation of the collection device 210 in a syringe-like manner and also reducing device operator contamination by providing intuitive physical barriers between the knob 216 and the distal end of the assembly device which holds the collection pad 218. Barriers formed by stops 230, 232 prevent the device from being placed too far inside the mouth, aid in positioning of the device in the mouth (vs. trying to hold a thin stalk), shield the user's fingers from being contaminated by the user's saliva, prevent anyone's fingers from sliding down too close to the pad, thereby potentially contaminating the buffer when the testor ejects the pad, they assist in the ejection of the pad by placing the index and middle finger of the user while allowing the user's thumb to operate the eject button (like holding a syringe) and assist in ejection by preventing the device from being placed too far inside the buffer tube during ejection. SVAI windows 224 may be placed around the circumference of the handpiece 212 so that the SVAI is viewable from all sides. In this embodiment, four SVAI windows 224 are positioned equidistantly around the handpiece 212. However, other numbers of SVAI windows and distances may be used. Pad holding notches 234 may be formed in the distal end of the handpiece 212 to receive and exert pressure on the clamps 220 and both sides of sample collection pad 218 to retain the pad 218 in the handpiece 212. One or more pad holding notches 234 may be shaped, for example, as a tear drop or other shape so that the distal most end of the notches 234 are closer together at the distal end than other corresponding locations along the length of the notches to maintain force on the clamp 220 and collection pad 218. In one aspect, clamp 220 may extend along approximately 10-50% of the length of opposing sides of the collection pad 218 in the assembled configuration, illustrated as attachment length 219. In one aspect, the clamps 220 may be longer and perforated so as to enclose the pad 218. The user inserts the clamped pad into the oral cavity for collection of the sample.

By positioning distal most ends of notches 234 closer together, a narrowing is formed to create a physical snap feature, wherein when the knob 216 is fully depressed to ejector stop 230, during a collection pad ejection, the physical snap feature creates a haptic feedback to the user, namely requiring a build of manual force followed by release with movement. In alternative embodiments, either the shaft of handpiece 212 proximal to the ejector stop 230 or the proximal surface of ejector stop 230 is formed with notches to create a similar physical snap haptic feedback to the user, when the knob is depressed to adequately eject the collection pad 218. In alternate embodiments, the haptic snap feedback sensation may be replaced or accompanied by an audible click sound during ejection. These feedback mechanisms provide signals to the user that the sample has been successfully ejected into the buffer tube, especially in poor visibility situations, such as roadside or low light conditions. In some embodiments, the handpiece 212 is formed from two identical molded or otherwise fabricated parts. The split line may be formed to be in line with the notches, or more preferably, moved to the face adjacent to each notch as illustrated. Optional reinforcing ribs 238 may be positioned along opposite sides of the handpiece extending from the distal end partially along the longitudinal axis of the handpiece at least the length that the pad holding notches 234 extend to provide additional strength to handpiece 212 in the region of the notches 234, if desired. The optional reinforcing ribs 238 may be formed along the split lines where the handpiece parts are joined to reinforce the notches 234.

As can be seen in FIG. 2B, inner shaft 214 extends beyond the length of the handpiece 212 and into the knob 216. Inner shaft proximal end 237 may be press fit into a recess 235 sized to receive the inner shaft proximal end 237. Wick 228 may be installed in a longitudinal channel 240 formed in the inner shaft 214 and further described in detail in FIG. 3. SVAI windows 224, 226, are aligned so that a proximal end of the wick 228 is visible through the SVAI windows. Prior to the activation of the SVAI, one color is visible through the SVAI windows. In some embodiments, the color shown is the natural color of the SVAI material or a shade of white or color. Distal end of the wick 228 is positioned and held in contact with the collection pad 218, via, for example, a press fit. Wick 228 may include a visual indicator, 242, for example a water soluble dye or water dispersible pigment at a location along the wick so that as saliva or other collected fluid collected in the collection pad 218 travels from the collection pad 218 and along the wick 228, the dye or pigment is transported with the saliva along the length of the wick, for example by capillary action. In this manner, the visual indicator 242 may become viewable through the aligned SVAI windows 224, 226 to the user. In one embodiment, the migration of the colorant into view of one or more SVAI windows causes a color change, indicating that the pad has absorbed a sufficient sample of oral fluid. Other indicator systems may be incorporated into an embodiment of the present disclosure. As one example, a porous polymer membrane or opaque fiber, such as Porex® brand materials, may be used where, upon wetting, the material becomes translucent thereby allowing a color strand to become visible. Visual indicator 242 could be any indicator system that allows users to observe a change through one or more windows (224/226).

In some embodiments, the wick may be made from one or more moisture-wicking materials such as from treated or untreated kraft softwood pulp, thermomechanical pulp, hemp fibers, cotton fibers, linen fibers such as Ahlstrom-Munksjo® grade 237 DNA/RNA blotting paper, specialty paper such as one or more of Grade 3511, Grade 8926, or Grade 11513 papers from EMI Specialty Papers, Whatman Papers grade 3MM, Ahlstrom® grade 320, Ahlstrom® grade 270, Cytiva® Grade 300 supplied by Cytiva Life Sciences, and various grades from Porex® Filtration Group, namely regenerated cellulose such as lyocell or Tencel®, rayon, cellulose derivatives such as cellulose acetate or cellulose triacetate, synthetic polymers such as PET, PETE, poly(acrylonitrile), poly(ethylene), poly(propylene), polyamides such as nylon-6, nylon-6,6, as supplied by Porex®, and other polymers and copolymers as well as blends thereof. One exemplary wick material is one that provides uniform absorption, and is available in a range of absorbency or thickness. A preferred absorbent material collects the liquid sample and transports it by capillary action through the material. As one example, cotton fiber material, such as Ahlstrom® 270 grade material may be used. In some embodiments, the wick may be pre-treated with a humectant solution, such as glycerol, or propylene glycol, and/or a non-ionic, anionic, amphoteric, or cationic surfactant to improve wicking speed and dried prior to application within the visual indicator.

In some embodiments, the visual indicator 242 may comprise a water soluble dye. Many different dyes may be used in the SVAI. One factor for consideration is water solubility. Another factor for consideration is FDA GRAS recognition, since the device will be inserted into the mouth of the user. Dyes such as C.I. Food Blue 2 (CI 42090, also known as FD&C Blue No. 1 and C.I. Acid Blue 9) or FD&C Yellow No. 5, or FD&C Yellow No. 6, tartrazine (also known as C.I. Acid Yellow 23) or FD&C Red No. 40, or FD&C Red No. 3, FD&C Green No. 3 (also known as C.I. Food Green 3). Alternatively, a water dispersible pigment colorant may be printed onto the wick. In some embodiments, the visual indicator may comprise an ink made of about 1-5% of a dye. In some embodiments, the visual indicator may comprise about 2-10% of an humectant, such as vegetable glycerine, 2-pyrrolidinone, or propylene glycol. In some embodiments, the visual indicator may comprise a preservative package such as about 1% phenoxyethanol, about 0.001-0.1% sodium benzoate, about 0.001-0.1% phosphoric acid, or Mickrokill® COS (Arxada), and about 92.5% water. As noted hereinabove, an aliquot of ink (approximately 5 microliters) may be printed at approximately halfway along the wick length so that the wick may be placed within the inner shaft 214 with either side of the SVAI being positioned at the one or more SVAI windows 224, 226. Placing the ink aliquot halfway along the SVAI wick is advantageous for assembly as the device is symmetrical along the length and may be inserted into the inner tube in either direction. Alternatively, the SVAI may be printed anywhere along the length provided that the ink is initially not visible through the window openings prior to activation, and becomes visible once liquids wick up the SVAI. Preferably, the SVAI is placed within a channel formed within the inner shaft 14 in such a way that the SVAI abuts against the end of the channel 250 while the distal end abuts against the pad 218 at the joint 252. The pad 218 may have a slightly curved surface at the joint 252 with the SVAI to enhance mechanical contact. Alternatively, the SVAI may contact the pad via an overlap in the two structures, or the SVAI may mate with a notch in the pad to ensure good fluid communication between the SVAI wick and the collection pad. The printed or stained section of the visual indicator 242 is not visible through the SVAI windows before the SVAI is triggered by sample collection.

The present disclosure is intended to include alternate methods to effect a sample volume adequacy indicator. As described herein, one function of the SVAI is a visual indication of when the collection pad has absorbed sufficient fluid for the analysis. Thus, any alternative indication method should be considered within the scope of the present disclosure. As one alternative, the collection pad and the SVAI may be manufactured in one piece from the same material using a roll die to stamp out the pad with integral SVAI from a sheet of absorbent material. The thin SVAI would then be printed with indicator ink and placed inside the inner tube assembly as described herein. Upon triggering, the combined pad and SVAI may be ejected into the buffer tube. Further, alternatively, a means to tear the pad from the SVAI may be incorporated. A frangible area may be incorporated on the clamp 220 or shaft 214 to allow for the pad 218 to be torn off from the device 210. Alternatively, the SVAI window may be integrated with the knob 216 by forming it from a transparent polymer, and allowing the SVAI to run all the way up into knob 216, so that it can be seen by the user during collection. Still further, the present disclosure includes indicators of volume adequacy without using a mobile colorant. In this regard, uncolored fibrous materials appear to be white because they have a high surface area and therefore scatter light readily at the interface between the fiber surface and the air. When fibrous materials become wet, the air is displaced by water, which has a refractive index much closer to that of the fibers—the scattering surface area effectively decreases, reducing light scattering. The material becomes more translucent as a result and heretofore hidden colored objects become apparent. One method to make an SVAI that works on this principle is marketed by Porex® Filtration Group, and comprises a high loft low density (0.05 g/cc-0.5 g/cc density range, or more ideally 0.075 g/cc to 0.20 g/cc density) lightly bonded fiber bundle formed into rods. These fibers may be made from any colorless polymer, including natural polymers, natural polymer derivatives, regenerated natural polymers, and synthetic polymers. The fibers may optionally be treated with a surfactant to enhance wicking. The fibers are oriented in a substantially coaxial direction to facilitate wicking along the length. A thick, darkly colored thread is included and hidden within the white fiber bundle. When saturated with liquid, the composite becomes translucent allowing the deeply colored thread to become clearly visible from outside the bundle.

In one embodiment of the collection pad 218 of the present disclosure, open cell foams may be used for the collection pad. Examples of open cell foams include poly(urethane) foams, poly(propylene), or melamine resin foams. Scintered polymeric materials may also be used. Such materials may be sourced from Porex® or Zefon®, such as Zefon PFSP37. Embodiments include cellulose, regenerated cellulose, cellulose derivatives, synthetic polymers, borosilicate glass, mineral wood, chopped glass fibers, or resin bonded chopped glass fibers, or mixtures thereof.

In some embodiments, the collection pad 218 may be made of cellulose, such as from treated or untreated kraft softwood pulp, thermomechanical pulp, hemp fibers, cotton fibers, linen fibers, such as Ahlstrom-Munksjo® grade 237 DNA/RNA blotting paper, Grade 3511, Grade 8926, Grade 11513 papers from EMI Specialty Papers, Whatman Papers grade 3MM, Ahlstrom® grade 320, Ahlstrom® grade 270, Cytiva® Grade 300 (supplied by Cytiva Life Sciences), and numerous grades from Porex® Filtration Group regenerated cellulose such as lyocell or Tencel®, rayon, or cellulose derivatives such as cellulose acetate, cellulose triacetate, or synthetic polymers such as PET, PETE, poly(acrylonitrile), poly(ethylene, poly(propylene), polyamides such as nylon-6, nylon-6,6, as supplied by Porex®, and other polymers and copolymers as well as blends thereof. Open cell foams may also be used for the collection pad, such as poly(urethane) foams, poly(propylene), or melamine resin foams. Scintered polymeric materials may also be used as collection pads. Such materials may be sourced from Porex® or Zefon®, such as Zefon PFSP37 as an example. Collection pad 218 and wick 228 may be made from a high purity cotton fiber pad material. One exemplary material is Ahlstrom® 320 grade cotton fiber. In some embodiments, the collection pad material has a greater density than the wick material. In some embodiments, the collection pad material has a lower density than the wick material. In some embodiments, the collection pad material has a greater void volume than the wick material, and in others it has a lower void volume. In further embodiments, the density and or void volume of the two components are the same.

As can be seen in FIG. 3A, inner shaft 314 can be formed from first 344, and second 346 body components. In some embodiments, the first 344 and second 346 body components are complimentary, so details described for the first body component 344 will be substantially the same for second body component 346. Along an interior wall 348, a wick receiving channel 340 is formed including sidewalls 352, an end wall 354, and one open end 356. Sidewalls 352 and end wall 354 may locate the wick, described above, so that it is visible through one or more SVAI windows 326 and prevent it from being positioned too far towards the inner shaft proximal end. Open end 356 may locate the wick so that it contacts the collection pad within the clamp portion of the dual lever clamp 320. The retention means may include a dual lever clamp 320 integrally formed with the inner shaft 314. Dual lever clamp 320 may include arms 358, 360 for holding the collection pad in the assembled position as seen in FIG. 2A. In other words, the dual lever clamp 320 may comprise two projections (or arms) in any configuration from the inner shaft 314. As illustrated, one embodiment provides arms 358, 360 of the clamp 320 may include one or more perforations 362 to improve the ability of the clamp to hold the collection pad (not shown in FIG. 3A) and to overcome adhesion or stickiness (e.g. due to, for instance, capillary action, or bridging by high tack fluids) upon ejection that might otherwise cause the wet pad to stick to the surface of the clamp arms 358, 360. The perforations also enhance fluid communication between the user's mouth and the collection pad. Additionally or alternatively, the arms may be smooth, textured, or shaped to enhance holding securely while in an assembled configuration, and/or ejection or release after collection, and/or the absorbing, including wicking, function of the collection pad.

The dual lever clamp portion, including the arms, of the clamp may include a wide range of textures, perforations, or other configurations. The clamp 320 may be integral with the inner shaft 314, or it may be a separate component. As illustrated in FIG. 3B, in one embodiment the clamp 320 is formed as a separate component, and has an opening 321 through which the inner shaft 314 may be thread. In this embodiment, clamp 320 may be formed from one sheet of material folded, with the opening 321 located across the proximal edge of the fold. If the clamp 320 is formed as a separate single sheet, then the inner shaft 314 preferably has lateral projections 322a and 322b that retain the clamp following ejection. In one embodiment, the length of the projections 322a and 322b are coextensive with the length of the fold edge 323a and 323b. Still further, alternatively the clamp 320 may comprise two arms that are affixed to the inner shaft 314. The material used for the clamp portion may be treated in various ways to enhance the grip on collection pad, whether the pad is wet or dry, both to ensure that the pad does not move during shipping and to ensure that it stays in place during collection in the mouth of the user. The treatment may also assist in the release of the pad into the sample tube containing buffer solution. The clamp material may be perforated. During perforation of plastics such as poly(propylene) and poly(ethylene), the perforating tool tends to draw material at the edge of the perforation cut into a raised area, consistent with the direction of the die used to perforate. If a very small die or needle is used to perforate the duck bill, the perforations may resemble small volcanoes with peaks following the initial penetrating direction of the needles when viewed in three dimensions. The perforations thus give the clamp differing frictional properties on each side. The volcano shapes formed by the die may assist in gripping of the sample collection pad, and further reduce sticking of the wet pad to the duck bill by reducing capillary action. The clamp surface may be roughened, or it may be polished smooth. As noted above, the surface of the clamp may be treated with a varnish that enhances release, or it may be coated to provide a micro texture. The clamp may also be embossed with a repeating pattern. The clamp may be molded with small features. Such features may include hooks, spikes, points, divots, or other shapes as may be desired.

In some embodiments, the clamp is formed into a generally open position prior to assembly of the device, so that the clamp naturally wants to spring open when the clamp and pad are pushed away from the clamp prongs 236, as shown in FIG. 2A. In this regard, as one example, the molded clamp arms 358 and 360 are formed non-coplanar with the remainder of the inner shaft 314, specifically each arm 358, 360 is non-coplanar with its respective body component 344, 346. The angle between the plane of the arm 358, 360 and the respective body component 344, 346 is between about 3° and about 90°. More preferably, the angle between the plane of the arm 358, 360 and the respective body component is between about 7° and about 60°. Still more preferably, this angle may be between about 10° and about 45°. In the case of embodiments that comprise a separate clamp, the clamp may be folded with an internal angle of about 5° to about 1750 prior to assembly, or unfolded (substantially flat) prior to assembly. In an unfolded configuration, the clamp may have preformed perforations for the user to accurately fold the clamp.

Other embodiments may include designs of retention means that do not include a clamp portion 320. Therefore, as shown in FIG. 3A, inner shaft 314 would not have arms 358, 360. For example, the collection pad is held in place solely by the clamp prongs 236, as shown in FIG. 2A. Once an adequate volume of sample is achieved, the user would push knob 216, which would advance inner shaft 214 to push the collection pad 218 from the clamp prongs 236.

FIGS. 4A and 4B illustrate alternative clamp embodiments for the collection device of the present disclosure. In FIG. 4A, a dual lever clamp 420 may be formed of a sprung ended thin, continuous piece of flexible material that is capable of elastic strain, with the sprung end having bracket-forming arms 463 biased away from each other in the illustrated release position and configured to receive, hold, and eject collection pad 418 similar to FIGS. 1-2. As illustrated in FIG. 4B, a clamp 420 may include indentations to meet with a profiled pinch point 464 provided along each arm 458 and may fit into corresponding protrusions 462 formed in the interior wall of the handpiece 412 to form an internal pinch fastener for holding the collection pad 418 securely, and similarly offering tactile response for ejection and release of the collection pad similar to that described hereinabove with FIGS. 1-2. The lever clamp could alternately comprise a flexible portion on one side and a stiff straight portion on the other side. In this way, only one flap of the duckbill opens to release the pad upon ejection. In some embodiments, the duck bill portion of the clamp may be omitted entirely.

In FIG. 4C, an inverting clamp 420 includes a flexible air or fluid filled clamp 466 having one end 468 coupled to and movable with the inner shaft 414 and an opposite end 470 coupled to the handpiece or an insert coupled to or formed with handpiece 412 to retain the collection pad 418. Flexible duck bill 466 may fully expand out of the inner shaft as seen in FIG. 4D via movement of the inner shaft 414 to eject the collection pad 418.

Another alternate embodiment may include a design in which the inner shaft includes a pin or hook at the distal end. As shown in FIG. 4E, the pin or hook 417 goes through a preformed perforation 419 of the sample collection pad 418, thereby holding the collection pad in place on the inner shaft 414. The SVAI wick pad abuts against the end of the collection pad. The pin or hook 417, as well as a portion of the collection pad proximal of the perforation 419, all retract into handpiece 412, with lateral cut-outs 413. When retracted, the pad 418 is unable to leave the pin or hook 417 due to a lack of clearance between the pad 418, the inner shaft 414 and the pin or hook 417. Once an adequate collection volume is reached, then the pad 418 may be removed from the mouth and the knob depressed to eject the pad. When the knob is depressed, the inner shaft 414 is pushed from handpiece 412 and the pad 418 and hook 417 is moved outside of the handpiece 412, where the pad 418 is allowed to fall into a sample collection vial, which preferably contains buffer solution. Optionally, a clamp may be added to this design to help secure the pad onto the inner shaft pin or hook 417.

In some embodiments, it may be desirable to collect saliva in two collection pads simultaneously. The two pads may be formed before, during, or after collection by separating a single pad. The single pad may be divided using a cutting blade or a knife formed from plastic or metal, or it may be torn apart along a pre-formed perforation, or a combination of both. Alternatively, two pads may be stitched or crimped together, collect saliva at the same time, and then unstitched or uncrimped to separate the pads.

FIG. 5 illustrates an oral fluid collection kit in accordance with the disclosed embodiments for the collection, preservation, and transport of oral fluid specimens. The kit 500 may include an oral fluid collection device 510 and a collection container, for example collection tube 572. Collection tube 572 may include a lid 574 and a buffer 576. Collection tubes may have various capacity and dimension. In some embodiments, tubes will have about a 5 to 15 mL capacity. The tubes may be made of poly(propylene) or other types of plastics and blends. The tube lid 574 may be push stopper, flip top, or a screw cap, where any configuration that retains the contents in the container in a fluid tight manner is acceptable. Collection tube 572 may contain a preservation buffer, for example about 3.0-3.3 mL of a preservation buffer resulting in an about 1 to 4 dilution factor of an about 1.0 mL or greater oral fluid sample. The collection tube may allow use of a variety of liquid handling automation, including filling with buffer, centrifugation, plunging, as well as automated sampling at the lab. The tube may have a flat or skirted bottom. Packaging 501 such as paper, low density poly(ethylene) (“LDPE”), Mylar, or biaxially oriented poly(propylene) (“BOPP”) may hold the device 510 and the collection tube 572, preferably with tamper evident security. Packaging may include a thermoformed blister pack with two internal chambers to contain the collection device and the collection tube, while providing physical separation of them in each of the two internal pack chambers. The buffer containing tube typically has a suitable taper, being slightly wider at the mouth than at the bottom. The taper, of for example 0.5° to 1.0° assists with removal of the tubes from an injection molding tool during manufacture. Buffer may be a neutral buffering system, or it could be an acid or a basic buffering system. One example is a neutral pH buffer containing a phosphate salt buffer, a detergent and a preservative. Additional ingredients may include polymers such as polyethyleneglycol, poly(vinyl pyrrolidone), chelating agents such as ethylene diamine tetraacetic acid, oxalic acid, citric acid, solvents such as ethylene glycol, propylene glycol, 1,3-propanediol, diethylene glycol and 2-pyrrolidinone, antioxidants such as ascorbic acid, ellagic acid, phenolic materials, and/or colorants such as dyes. DNA and RNA preserving agents such as DNASE or RNASE inhibitors or reagents to inhibit proteases may also be included. Preferentially, the buffering system preserves the sample for storage and/or analysis. Any buffer system may be used for one or more of preserving the same and facilitating the extraction of analytes from the pad, such as a citrates, TRIS (tri-hydroxymethyl aminomethane), Bicine buffers. Other buffers may be used to preserve the saliva along with analytes present. Published Chinese patent application CN 105039306A discloses, for example, a water based saliva preserving buffer system for nucleic acid analysis comprising:10-50 mmol·dm⁻³ tris (tri-hydroxymethyl aminomethane)
0.5-20 mmol·dm⁻³ Ethylenediamine tetraacetic acid, sodium salt,
200-1000 mmol·dm⁻³ Ammonium sulfate
1-50 mmol·dm⁻³ Glucose.

NASA disclosed a useful preservative buffer for preserving astronaut saliva samples without the need for refrigeration (NASA Tech Briefs, August 2012, accessed on line Jun. 27, 2022) This preservative buffer technology includes:

| | |
|---|---|
| Sodium dodecyl sulfate | 0.5% |
| EDTA (ethylene diamine tetraacetic acid) | 1 mmol · dm⁻³ |
| Tris (tri-hydroxymethyl aminomethane) | 1 mmol · dm⁻³ |

Although the kit is illustrated with a single fluid collection device and collection tube, multiple collection devices and/or collection tube may be provided in a kit. While a bag is shown in FIG. 5, any kit container may be used, including but not limited to a blister pack, a clear box container, a larger tube, or a zippered pouch. Preferably, the kit includes an insert preprinted with instructions for use. The final kit may be used for single use (single pad/tube) or for split-sample collection (i.e. single device with continuous single pad that may be split post-collection or single device that holds multiple pads and multiple buffer tubes).

In a method of fluid collection, an oral fluid collection device is inserted with the collection pad leading into an oral cavity 602. The collection pad may be positioned between the cheek and gum and/or under the tongue. Oral fluid may collect in the pad until the pad is full and then may travel up the wick. The collection pad is maintained in the oral cavity 604 and the SVAI is checked to see if sufficient fluid has been collected 606. An additional feature of the design is that the SVAI can be easily checked while the device is working in the oral cavity. Multiple SVAI windows 224 allow viewing from a range of angles. The guard feature 232 prevents the wand from being located too far into the mouth, in this way the SVAI is visible outside of the mouth.

Preferably, the device is not removed until the SVAI offers a signal that an adequate volume has been collected. If, however, a visual indicator does not offer a signal for adequate volume collection, then sufficient fluid has not been collected and the device and collection pad may be maintained in the oral cavity 604 for a predetermined amount of time, such as 15 minutes. If the visible indicator does appear, then sufficient fluid has been collected and the device may be removed from the oral cavity 608. The visible indicator may be, for example, a color indicator appearing in the SVAI windows. The device may be configured so that the visible indicator appears once at least about 1.0 mL or greater (±10%) of oral fluid is collected. However, the SVAI may be designed to indicate collection of either more or less fluid volume. In some embodiments, the device is configured to collect an adequate sample volume of oral fluid within about two minutes of insertion into the mouth. As noted hereinabove, however, different users may experience shorter or longer times. In other embodiments, the SVAI may trigger only after 2.2 ml of fluid has been collected in the pad, or the SVAI may intentionally trigger at much smaller oral fluid amounts such as 0.7 ml.

Once the device is removed from oral cavity, the pad with the fluid sample may be decoupled or released from the device for placement into the collection tube 610 so that no parts of the device, such as plastic parts of the inner shaft, clamp, or handpiece, are released with the collection pad. In other words, in a preferred embodiment, only the collection pad is deposited in the tube. The tube may be sealed with a cap and the device discarded or recycled. As an example of the ejection operation, the user may place their thumb on top of the handle and press the knob towards the ejector stop to eject the collection pad. Inner shaft moves distally in response to the knob pressure and the clamp arms expand, or move away from each other, to release the collection pad. In this manner, a user may operate the device to eject the collection pad with one hand while holding the collection tube in the other hand allowing for collection in a variety of environments including roadside as well as office environments. In some embodiments, the handpiece may be sized so that the distal end fits in the interior of the collection tube, and the user rests the end stop of the hand piece on the collection tube during the ejection of the collection pad. The collected sample may further have appropriate labels applied and be sent to a laboratory for testing. In one embodiment, the oral fluid collection kit may allow for the collection, preservation and transport sufficient oral fluid specimens for testing for drugs of abuse such as tetrahydrocannabinol (THC), benzoylecgonine, cocaine, morphine, codeine, oxycodone, oxymorphone, hydrocodone, hydromorphone, 6-acetylmorphine, phencyclidine, amphetamine, methamphetamine, MDMA and MDA. Other analytes found in oral fluid may optionally be collected and tested, including but not limited to whole cells, DNA, RNA, steroids, proteins, microorganisms, viruses, fungi, vitamins, metals, hormones, metabolites, blood, and the like. Therapeutic drugs may also be analyzed using oral fluid to ensure distribution throughout the body. Examples of therapeutic drug classes may include antibiotics such as oxytetracycline, vancomycin, azithromycin, anti-viral compounds such as emtricitabine, tenofovir disoproxil fumarate, peginterferon alfa-2a, anti-fungal compounds such as Diflucan, ketoconazole, and other therapeutic drugs.

In a method of manufacturing, the handpiece components are formed 702, for example by injection molding. In some embodiments, handpiece components include the clamp. In some embodiments, knob may be formed 706, for example by injection molding. Inner shaft components, or a pusher are formed 704. In some embodiments, the inner shaft is formed or injection molded with a dual lever clamp integral to the inner shaft components. In some embodiments, the dual lever clamp is manufactured separately and coupled to the inner shaft components. For example, the inner shaft components may each have an opposing projection and the dual lever clamp may have an opening that slides over these projections when the inner shaft components are assembled. Alternatively, the inner shaft and the ejection knob may be molded as one piece.

The clamp may be made from a polymeric material or, alternatively, a thin metal. The dual lever may be made from a single sheet and folded, or from two discrete parts welded, clamped, or otherwise coupled together. Arms of the dual lever may be sized, textured, and/or perforated, or modified in some other way to facilitate gripping of the pad while in the mouth, and release of the pad when ejecting. For example, the wet pad may cling to the dual lever during ejection by way of stickiness of the wet pad against a smooth surface, (due to e.g.: capillary action) if the arms are too smooth and large, whereas perforations or texture added to the walls may permit airflow between the wet collection pad and the clamp arms to release the wet pad.

Wick is formed 708. For example, a visual indicator is deposited in the middle of the wick and dried. The dried wick is cut to fit the device dimensions. In some embodiments, the indicator is deposited by printing such as ink jet (continuous, thermal drop-on-demand, piezo drop-on-demand), micropipette dispensing, direct gravure printing, anilox metered roller coating, flexographic printing, pad printing. While depositing the water soluble ink mid-length of the SVAI wick has some advantages, the ink may alternatively be placed to one side or another of the SVAI mid-length point. So long as, once assembled, the color is not visible before the pad is saturated and becomes visible when the SVAI is triggered. In some embodiments, the ink used for printing the visual indicator Ink used for printing may include a water soluble dye, and optionally: an humectant to reduce crystallization, drying, crusting, and clogging of printing nozzles, a surfactant to control surface tension, viscosity and rheology modifying agents, preservative(s), a corrosion inhibitor to help protect printing equipment or other systems like Porex® translucent fiber or colored membrane. The wick may also be formed by slitting down a roll of an absorbent roll good into multiple thin strings. These strings may be printed, dried, and positioned within the inner shaft before cutting to length. In some embodiments, the SVAI wick may be formed using a die to cut the long thin shape from a pad of material, or alternatively cut using a sharp knife such as a razor blade.

Wick is assembled into the inner shaft or pusher 710. In particular, the wick may be placed in a channel or recess of the inner shaft component. In some embodiments, inner shaft components are fitted together. Inner shaft and wick assembly is coupled to the handpiece 712. In some embodiments, shaft may be inserted into the already assembled handpiece starting at the distal end. In some embodiments, the handpiece may be assembled around the fitted inner shaft. For example, the inner shaft, or pusher may be positioned in one part of handpiece and the other part of the handpiece may then be coupled to the first part. Handpiece parts may be assembled by attaching them using crush pins, ultrasonic welding, clamping, or adhesive welding. In some embodiments, the inner shaft is held together by the insertion into the outer handpiece. In some embodiments, the inner shaft is fitted together and attached using crush points, ultrasonic welding, clamping, or adhesive welding. Mutual notches or other projections on the interior walls of the inner shaft components prevent independent co-axial motion during ejection. Knob may be attached to a proximal end the coupled handpiece and inner shaft 714. The knob may also assist the crush pins in holding the inner tube sections together.

Collection pad is formed 716 by cutting, for example cut from a larger pad using a die, or a rotating blade, or a guillotine. The sample pad may be cut and assembled into the inner shaft prior to inserting the inner shaft into the handle and attaching the knob. In some embodiments, the collection pad may be rectangular, or trapezoidal for ease of insertion into a collection tube. For example, the wider part of the trapezoidal pad may be substantially the same width as the tube diameter and be mounted to the clamp to be inserted last. In yet other embodiments, the pad may be an irregular shape or shaped like a bullet. The wick and collection pad may be made by die-cutting, injection molding, forming, sintering, casting fiber onto a wire. In some embodiments, the wick and collection pad may be formed as a single unitary structure. The collection pad may have curved edges, so that oral irritation of the cheek is reduced. In addition, adding a concave radius to the distal end of the pad may improve fluid communication between the SVAI and the pad at the butt joint.

Collection pad is detachably coupled to the inner shaft assembly or the handpiece 718. In particular, collection pad may be placed within clamp arms of the inner shaft or the handpiece to form an interference fit (butt joint) with the wick. In some embodiments collection pad may be mounted to projections or a lock and key mechanism in the handpiece at the time the pusher is placed in one side of the handpiece. In some embodiments, shaft may be inserted into the already assembled handpiece starting at the distal end. In some embodiments, the handpiece may be assembled around the fitted inner shaft. The method may end at the formation of the device or continue to form a kit in FIG. 8. The pad and wick may alternatively be formed as one piece.

During kit formation, the fully assembled device may be packaged 820. A collection tube is filled with a buffer and capped 822. Collection device and collection tube are placed in kit packaging 824. A sample mailing pouch may also be included within the kit pouch. In some embodiments, the packaging may be designed so that it is re-usable as a container in which to double pack and ship the collected oral fluid sample. Alternatively, the pouch could be reused to gather and dispose of single use items contained in the kit.

Handpiece and/or inner shaft may have circular cross sections. However, in some embodiments the handpiece and/or inner shaft may have a cross sectional shape of a square prism with rounded edges to maintain the orientation of the inner shaft so that mutual rotation of the handpiece and the inner shaft is not possible. Both the handpiece and inner shaft may be made in two pieces, handpiece, inner shaft, and knob may be made by injection molding, blow-molding, or 3-D printing. They may be made from plastic such as poly(propylene), poly(ester), poly(styrene), ABS (poly(acrylonitrile-co-butadiene-co-styrene)) or a variety of thermoforming and thermosetting polymers. Other materials may be used, such as glass or paper or wood, where the components may be pressed, formed, stamped, cut, 3D printed, or carved.

In some embodiments, a butt joint between the pad and the wick may be used to provide a degree of flexibility for lateral rotational movement of the pad within the duckbill clamp. In this manner, the pad may be laterally displaced off the device longitudinal axis by around 30°, and still will absorb liquid to trigger the SVAI. This property may help the comfort of a fluid donor within their mouth. In some embodiments, the wick may overlap the pad to increase the contact surface area. In some embodiments, the pad may have a cutout portion and the end of the wick may be shaped to fit into the cut-out portion to increase the surface area contact between the pad and the wick, and may be secured in contact via staples or stitches. The disclosed couplings between the pad and SVAI wick ensure the pad remains in good fluid contact with the SVAI wick during shipping and handling prior to sample collection in the mouth. The pad must not come loose through vibration, or when the pad is being placed between the cheek and gum. Disclosed embodiments also hold the pad firmly in the clamp for the duration of the sample collection to ensure that the SVAI is triggered appropriately, and when removing the assembly from the fluid donor's mouth, as there is a tendency for pads to stick to the cheek or gums during oral fluid donation. Conversely, once the SVAI has been triggered, and the assembly removed from the mouth, the saturated pad ejects smoothly into the buffer tube. For this reason, the force needed to pull the dry pad from the assembly (without pressing the eject button, or knob), as well as the force needed to pull a saliva saturated pad from the assembly are important characteristics.

Forces, in particular pull strength in a vertical direction, required to dislodge a sample pad from the disclosed duckbill clamp may be determined using the Dry Pad Retention Strength Protocol for a dry pad assembled collection device, and the Wet Pad Retention Strength Protocol when the pad saturated so the SVAI visual indicator is visible in the SVAI windows of the collection pad device. Results of weight and/or force applied at the point the pad fully detaches from the assembled duckbill clamp collection device are shown in Table 1 for toothed and smooth perforated clamp surfaces. The tests were performed by suspending the handpiece vertically, for example via a clamp, and attaching a basket to the saturated and/or dry pad for weight to be added incrementally until the pad fully detached from the duckbill clamp. Alternatively, a force gauge may be used to test the forces.

In particular, injection molded prototypes were assembled with die-cut absorbent pads and SVAI wicks, and tested using these test methods:

### Dry Pad Retention Strength Test Protocol

### Materials:

Assembled prototype devices.
A weigh boat or equivalent sample container such as a petri dish with a vessel wall no higher than 1-2 cm tall. For consistency purposes identify the vessel in testing notes and use consistently for all subsequent experiments. A reproducible device or pad holder should be contrived and used consistently though out the course of testing.
Gravimetric analytical balance capable of reproducibly measuring 1.0-2.0 g with accuracy of +/−0.001 g.
Timer/Stopwatch that is accurate to within +/−1 s.
Appropriate PPE, specifically, safety glasses or face shield and disposable gloves.
Scale calibration weight set (variable weights up to 1000 g)

An apparatus that will suspend the handle in a vertical position such that known weights can be applied to the pad for strength testing. Record mass of empty basket.

Protocol: Assemble the pull strength apparatus on a flat, firm surface. Ensure that the attachment points are secure and that the devices will be held in a vertical position, with the pad at the bottom. Attach the handle portion of the device to the apparatus so clamping mechanism hold the handle between the knob and the upper finger guard. Ensure that the device is not held too tightly to cause a deformation of the plastic. Also ensure that the knob is not able to move. Attach the weight basket to the bottom of the pad. Ensure that the weight basket is centered and aligns with the long axis of the handle. Gently place the weights into the basket. Avoid dropping weights into the basket. Avoid swinging or rotating of the weight basket. Incrementally add additional weights until the pad fully detaches from the device. Record the exact weight needed to detach the pad from the device. Calculate the following metrics using the following formulas and report: Adjusted mass (g)=mass of empty basket (g)+mass required for pad detachment for each device tested (g). Calculate mean and standard deviation of adjusted mass required for pad detachment for the population of devices. Assume 9.8 Newton force=1 kg mass.

### Wet Pad Retention Strength Test Protocol

### Materials:

Saliva sample, or artificial equivalent.
Only use if previously stored at −20° C. and within expiration identified during manufacture. Sample should be clear to slight hazy but no significant solids evident. Discard and request immediate replacement if microbial growth (smell, color, etc.) or significant precipitation is evident.
a. Assembled prototype devices. A weigh boat or equivalent sample container such as a petri dish with a vessel wall no higher than 1-2 cm tall. For consistency purposes identify the vessel in testing notes and use consistently for all subsequent experiments. A reproducible device or pad holder should be contrived and used consistently though out the course of testing.
b. Gravimetric analytical balance capable of reproducibly measuring 1.0-2.0 g with accuracy of +/−0.001 g.
c. Timer/Stopwatch that is accurate to within +/−1 s.
d. Appropriate PPE, specifically, safety glasses or face shield and disposable gloves.
e. Scale calibration weight set (variable weights up to 1000 g)
f. An apparatus that will suspend the handle in a vertical position such that known weights can be applied to the pad for strength testing. Record mass of empty basket.

Protocol: Assemble the pull strength apparatus on a flat, firm surface. Ensure that the attachment points are secure and that the devices will be held in a vertical position, with the pad at the bottom. Fill Weigh boat or sample vessel with non-expired artificial saliva to a height of no greater than 0.25 cm. Place a clamp stand close by to support the prototype in a vertical position. Dip unused prototype collection device pad into the saliva in the weigh-boat. Use the clamp stand to gently hold the device upright. Once the SVAI is triggered, then the pad will be saturated and can be transferred to the pull test rig. Attach the handle portion of the device to the apparatus so clamping mechanism hold the handle between the knob and the upper finger guard. Ensure that the device is not held too tightly to cause a deformation of the plastic. Also ensure that the knob is not able to move. Attach the weight basket to the bottom of the pad. Ensure that the weight basket is centered and aligns with the long axis of the handle. Gently place the weights into the basket. Avoid dropping weights into the basket. Avoid swinging or rotating of the weight basket. Incrementally add additional weights until the pad fully detaches from the device. Record the exact weight needed to detach the pad from the device. Calculate the following metrics using the following formulas and report: Adjusted mass (g)=mass of empty basket (g)+mass required for pad detachment for each device tested (g). Calculate mean and standard deviation of adjusted mass required for pad detachment for the population of devices. Assume 9.8 Newton force=1 kg mass

It is important that the absorbent pad remains clamped into the collection device during shipping, while being opened by the person wanting to collect the sample, and during sample collection. The pad must also stay on the collection device when withdrawn from the mouth, and furthermore not drop onto the floor under the influence of gravity before ejection into the sample tube. While difficult to measure, dry pads pressed onto the wet inside cheek do have some adherence to the cheek lining. By attaching thin wire to pads, adhering them to the inside cheek of a human, and then adding weights to the wire, we found that the approximate minimum force needed to dislodge a pad from an inside cheek surface to be about 50 g—or ˜0.5 Newton. This sets an absolute lowest minimum for the pad retention force, wet or dry, in that forces acting within the mouth will subject the pad to at least half a Newton, just by taking the pad out of the donor's mouth. The forces needed to dislodge the pad from an early prototype device are presented in Table 1. In this case, prototypes were made by injection molding a two part thermosetting resin into a 3-D printed mold, a process known as Pro-Jet.

TABLE 1

| | Mass to remove | Force to remove | Mass to remove | Force to remove |
|---|---|---|---|---|
| Prototype ID | dry pad/g | dry pad/N | wet pad/g | wet pad/N |
| 1 (Pro-jet | >270 g | >2.646N | >270 g | >2.646N |
| toothed | | | | |
| duckbill | | | | |
| (“PJTD”)) | | | | |
| 2 (Pro-jet | 240 g | 2.352N | 200 g | 1.96N |
| Smooth but | | | | |
| perforated | | | | |
| duckbill) | | | | |
| 3 PJTD | >350 g | >3.43N | 150 g | 1.47N |
| 4 PJTD | NT | NT | 130 | 1.274N |
| 5 PJTD | NT | NT | 150 | 1.47N |
| 6 PJTD | NT | NT | 190 | 1.862N |
| 7 PJTD | NT | NT | 160 | 1.568N |
| 8 PJTD | NT | NT | 240 | 2.352N |
| 9 PJTD | NT | NT | 160 | 1.568N |
| 10 PJTD | NT | NT | 270 | 2.646N |
| 11 PJTD | NT | NT | 240 | 2.352N |

Still other embodiments may include designs of retention means that are integral to the handpiece. As illustrated in FIG. 9A, first handpiece component 911a and second handpiece component 911b each coupled to a pad 920a, 920b. First and second handpiece components have a coupling mechanism, such a setoff corresponding projections and recesses 923a, 923b that may be separably connected to form handpiece 912 as seen in FIG. 9B for fluid collection and a unitary pad 920. Likewise, pads 920a, 920b may be separably connected, formed as a single pad folded in half, or formed as a single separable pad. After fluid collection first and second handpiece components 911a, 911b and respective saturated collection pads may be separable to place in separate collection containers. Each handpiece component 911a, 911b may be dragged perpendicular across the open end of the container when the pads 920a, 920b are inserted to severe the pads 920a, 920b, from the handpiece components 911a, 911b, for example at a weakened portion near the handpiece component 911a, 911b. Pads 920a, 920b may be joined using perforations to enable tearing of strips along the long edge. Alternatively, pads may be a single pad joined by smaller joining structures, which may also be perforated. Pads may be joined by dissolvable thread, which may include gelatin threads, starch threads, cellulose derivatives such as carboxymethyl cellulose, hydroxypropyl cellulose, poly(acrylic acid) and others. Alternatively, a tear-away stitched strip may be used that releases the two pads from each other when the end of the strip is pulled along the length of the two pads. FIGS. 10A-10D illustrate O-shaped 1020a, U-shaped 1020b, reverse U-shaped 1020c, and reduced width U-shaped 1020d single collection pads that are frangible along the reduced areas of width 1023 as a result of this reduced dimension or due to perforations or other localized reductions in strength to separate into two collection pads 1021a, 1021b after fluid is collected.

In one embodiment, a single-use device is made to simultaneously collect a total of at least 2 ml of undiluted (neat) oral fluid, that can be subdivided in the employee's presence, into an “A” and a “B” split sample of at least 1 ml±10 percent undiluted (neat) oral fluid per each included specimen bottle; or a single-use device made to simultaneously collect a sufficient amount of oral fluid, which can be subdivided in the employee's presence, into an “A” and a “B” split sample sufficient for laboratory testing. For example, when two specimens are collected simultaneously using a single collection device that directs the oral fluid into two separate collection tubes; or when a device collects a specimen with a single pad, which can be subdivided into two separate collection tubes. In this way, the two saliva samples are collected as one, then subdivided in the saliva donor's presence.

The single pad may be separated into two halves at the midpoint via perforation that ensures two identical halves when separated (see FIG. 11). A single pad may be folded either on long or short edge with perforations at the mid-point to allow consistent separation when force is applied to the mid-point, or opposing ends.

The folded halves may be cut or split evenly via manual pulling force or via pressure applied at the mid-point by the collection device or accessory. Alternatively, the single pad can be separated into two halves via a small interconnection between two larger sample collection sections (H-shaped, nun chuck shaped or dumbbell shaped).

The interconnection may be made of the same single piece of absorbent material and allows the pads to be separated via pulling, tearing, splitting apart into two unique samples. The single pad may be split into two halves when placed into a separation apparatus (for example, a device handle that applies force the mid-point (perforation, interconnection, etc.), or a unique sample tube with bladed separation element that applies force to the midpoint, perforation or interconnection and separates the single pad into A & B sample chambers.

Proposed device allows a single pad to be placed into the donor's mouth at the same location during a single collection event. This ensures both halves of the pad will experience similar conditions in the mouth during the collection event. The pad is subsequently subdivided in the donor's presence, as opposed to inserting two single use devices in the mouth on either side, or in each cheek, or consecutively. Proposed device comprises a single collection pad, ensuring after the single pad is split into two halves, the halves will have similar physical and chemical properties and will generate more consistent and representative testing results.

As seen in FIG. 11, collection pads 1120a, 1120b are separable at a pre-determined position 1127, converting the one pad 1120 into two pads 1120a, 1120b, following sample collection. sample split line 1127 at the predetermined position could be deeply perforated, or it could include a partial cut. The sample separation line could also include the use of dissolvable thread. Push rod 1131 may be slidably movable in the handpiece 1112 apply force to the sample split line 1127 when handpiece 1112 is pulled in the direction of the arrow in order to convert the one pad 1120 into two pads 1120a, 1120b. Handpiece may subsequently be pulled apart to form separate components separably couplable handpiece components 1111a, 1111b.

Single collection pads may be formed of cellulosic sheets, which are anisotropic. Typically, cellulose pulp fibers are laid down onto a porous forming strainer (“wire”) from a low solids slurry of ˜0.3%-0.6% by dry weight of fibers. The fibers are essentially strained out of the slurry by a wire in the wet end of the machine, which is called a fourdrinier. During this process, fibers tend to align with the flow of the slurry as well as the movement of the wire. Thus, the number of fibers generally oriented in the machine direction is greater than the number of fibers oriented in the cross-direction. This orientation means that mechanical properties of the fibrous pad produced are different in the machine and cross directions.

Pads made using other forms of papermaking also lead to anisotropy: Hydroformers inject one or more dilute pulp streams between a felt blanket and a roller to form the paper sheet. Some fiber alignment occurs as the material flows through the nozzle and is injected into the nip. When tearing a sheet by pulling in the machine direction, fibers tend to be aligned and slide over one another to make a fuzzy edge. When tearing a sheet by pulling in the cross direction, fibers are aligned perpendicular to the tearing force, leading to cleaner separation of the two sheets. Another point of note regarding orientation of the paper cut is that the “tearing force” required is significantly less in the machine direction than the cross-direction. In this regard, there is a balance, and these qualities may be used purposefully depending on the application performance desired. Other factors that influence tear strength include fiber length—generally, the longer the fiber, the higher the tear strength.

FIG. 12 illustrates another embodiment of a collection device with handpiece 1212, and collection pad 1220 extending into handpiece 1212 having a reduced diameter “d”. Collection pad 1220 further has a frangible point formed as a circumferential recess, or cut 1219. Collection pad 1220 may be formed as a single pad separable in to two pads 1220a, 1220b or as two pads joined together along plane 1217 as described with respect to FIGS. 9-11. Sample pad 1220 pad may be split along plane 1217 upon insertion into a divided container 1272 along a divider wall 1271 extending across the diameter and along the length of the container. The handpiece may also be separated snapped off at the frangible point from the pads once the sample pads are pushed home into the sample container 1272.

FIGS. 13A-13B illustrate a collection device in the assembled collection position and during separation. Two pads 1320a, 1320b coupled to respective handpiece components 1319a, 1319b are interlocked orthogonally, and used to absorb sample saliva. The pads and respective holders are separable with a sliding action in opposite directions as indicated by arrows in the long axis.

According to FIG. 14, a cross sectional view of a handpiece 1412 of another collection device may include flared distal end, or hood 1415 sized to receive and configured to retain pad 1420. In this embodiment the mechanism for retaining the pad 1420 includes protrusions 1435 extending radially inward inside the hood 1433 such as ridges, bumps, or teeth. The hood 1433 may be completely enclosed or have slots extending up the sidewall of the hood 1433. Slidable inner shaft 1414 may optionally include a proximal knob 1416 and/or distal enlarged end to apply even force to pad 1420 so that movement of the inner shaft 1414 towards the collection pad 1420 free the collection pad 1420 from the retaining mechanism. Although illustrated as solid, inner shaft 1414 may be hollow to accommodate SVAI wick and SVAI windows as shown and described in FIG. 1.

In the illustrative embodiment as shown in FIG. 15, a partial exploded view of an oral fluid collection device 1510 having a handpiece 1512, an inner shaft 1514 forming a pusher 1511 and a knob end 1516, and a sample collection pad 1518 is shown. Inner shaft 1514 is configured to be inserted or otherwise connected to handpiece 1512. Retention means, such as clamp 1520 holding the pad 1518 in place during shipping and fluid collection at the distal end of handpiece 1512 may exert a gripping force perpendicular to the plane of the sample collection pad 1518. The pad 1518 is lined on either side with clamps 1520, preferably formed of polymeric materials. These clamps 1520 may be fixed claims with radially inner portions that are flexible clamps 1521. Flexible clamps 1521 may be configured to expand outward from a neutral position as the pusher 1511 is pressed to push collection pad out of the handpiece 1512. The overall configuration enhances the grip on the pad 1518, as well as participates in the release of the pad 1518 at the end of the collection process. As described further in FIGS. 16A-B, clamp 1520 at the distal end of the handpiece 1521, in particular a fixed clamp 1520 may hold the sample collection pad 18 in the assembled position during shipment and fluid collection, and release the sample collection pad 1518 via the radially inner flexible claims 1521 from the handpiece 1512 after fluid collection is completed.

Handpiece 1512 can be formed from first 1513a, and second 1513b body components. In some embodiments, the first 1513a and second 1513b body components may be nonmirrored, mirrored, or complimentary, so details described for the first body component 1513a will be substantially the same for second body component 1513b. Second body component 1513b forms an arm that has fixed clamp 1520 and flexible clamp portion 1521 at a distal end. Fixed clamp 1520 radially surrounds flexible clamp portion 1521 and includes pad holder projections 1531 that extend into fixed clamp opening, sized to receive the collection pad 1518 at a fixed dimension. For example, fixed opening may have a width greater than the collection pad with, in the range of 0.1 to 10 mm, for example 6 mm depending on thickness of the pad. Fixed clamp 1520 may also include inner and outer guidewall ramps 1533, 1534 along a portion of the length of the arm 1513b to guide inner shaft 1514 of pusher 1511. An inner guidewall ramp 1533 may extend from an SVAI window 1524 in a negative slope towards and terminate at a key of a locking component. Additional inner guidewall ramp 1533 may be formed at an end of the handpiece 1512 opposite the arms 1513a,b to serve as a stop and prevent further pushing of the knob end 1516 in a longitudinal direction. Guidewall ramps may 1533 be serrated, for example as seen and described with respect to FIG. 18D, or otherwise textured to hold a wick counter to the ejection force.

Flexible clamp portion 1521 may include release ramps 1536 and a pad locking component 1537. Pad locking component 1537 includes a convex projecting key 1538 on one flexible clamp portion 1521, and a corresponding opening or lock 1539 sized to fit the key. Pad locking component may be oval as illustrated or other complimentary shapes. Pad locking component may utilize a spot adhesive on the key mating surfaces of 1538 bind the pad holding features to ensure the device does not eject the pad prior to use. Release ramps 1536 are configured to interact with corresponding ramped walls 1540 on inner shaft 1514 of pusher 1511 to cause force to open flexible clamp portions 1521, separate the lock 1539 and key 1538, and release the collection pad 1518 as knob 1516 is pushed. This may create a haptic feedback while overcoming the friction of the release ramps 1536. Additionally, release ramps 1536 may reduce unintended release of the pad due to accidental depression of the pusher. Release ramps 1536 may have an angle of incline from the longitudinal plane of the clamp surface of in a range of 15-45 degrees, and preferably 35 degrees.

Pusher 1511 may include a knob end 1516 with inner shaft 1514 formed as two longitudinally extending parallel arms 1548 with a recess or channel 1529 formed between the arms that is closed at one end by the knob end 1516 and open at the opposite end. Arms 1548 each include a ramped wall 1540 extending along its length on both sides of each arm 1548 and terminating prior to the distal end of each arm at a same position. Release ramps may have a sloped positive angle followed by a flat top so that the corresponding sloped surface of the ramped wall 1540 so that the force applied to the knob 1516 may move the sloped surfaces along each other until the maximum opening the flexible clamp 1521 is reached at the flat parallel flat tops of the release ramps 1536 and ramped walls 1540. Beyond the terminal end of each ramped wall 1540, each arm 1548 has an enlarged flat push contact surface 1550 for contacting and pushing collection pad 1518 out of the handpiece 1512. In an assembled position, outer guidewall ramps 1534 are located outside of arm 1548 of pusher 1511, and inner guidewalls 1533 align with the interior side of the ramped wall 1540 of the pusher 1511, which also may align with the recess 1529. Guidewall ramps 1533, 1534 and stop 1507 cooperate with movement of the knob 1516 towards the ejector stop 1507 during motion to eject the sample collection pad 1518, allowing for single-handed operation of the collection device 1510. Pusher may travel a range of 1-50 mm to open the clamp, or 2-25 mm, or 2-3 mm. After the pusher has opened the clamp, or jaw, the pusher may travel 1-100 mm, or 10 mm to eject or release the pad from the device 1710. The force required to depress the pusher may be in a range from 0.1-10 lbs, for example 2 lbs.

A sample volume adequacy indicator (SVAI) 1522 may include one or more sample volume adequacy indicator windows 1524, that align with each of the handpiece 1512 and as well as a SVA1 wick 1528 that may signal a user visibly through the one or more SVA1 windows 1524, when a sufficient volume of oral fluid has been collected. SVAI wick 1528 may be coupled in a recess 1529 in pusher 1511, for example by a friction or interference fit so that wick 1528 protrudes from the end of pusher 1511 in a direction towards a notched section of collection pad 1518.

Collection pad 1518 may include a notched section 1551 at one end sized and shaped to accommodate the end of the SVAI wick 1528 and a lock opening 1552 sized and shaped to form part of pad locking component 1537. Lock opening 1552 aligns with lock 1539 so that the key 1538 extends through both the lock 1539 and lock opening 1552 in the locked and assembled position.

Referring to FIG. 16A, an exemplary embodiment of an assembled oral fluid collection device 1610 is shown. The fixed clamps 1620 and flexible clamps 1621 are shown assembled and holding a sample collection pad 1618 with pad locking component 1637 key extending through lock and lock opening in the collection pad 1618. Pad holder projections 1631 extend towards each other on opposite side of collection pad 1618 to provide additional stability to the pad 1618 in the assembled position. The handpiece 1612 includes an opening through which knob portion 1616 of pusher extends out of handpiece 1612, with the SVAI windows 1624 positioned between the knob 1616 and fixed clamp arms 1620. SVAI windows 1624 may be placed around the circumference of the handpiece 1612 so that the SVAI is viewable from all sides. In this embodiment, two SVAI windows 1624 are positioned equidistantly around the handpiece 1612. However, other numbers of SVAI windows and distances may be used. As illustrated in FIG. 1613, SVAI wick 1628 contacts collection pad 1618 in the assembled position and collection pad 1618 is secured via pad locking component 1637

In the illustrative embodiment as shown in FIG. 17, a partial exploded view of an oral fluid collection device 1710 having a handpiece 1712, an inner shaft 1714 formed of a pusher 1711 and a knob end 1716, and a sample collection pad 1718 is shown. Inner shaft 1714 is configured to be inserted or otherwise connected to handpiece 1712. Retention means, such as clamp 1720 holding the pad 1718 in place during shipping and fluid collection at the distal end of handpiece 1712 may exert a gripping force perpendicular to the plane of the sample collection pad 1718. The pad 1718 is lined on either side with clamps 1720, preferably formed of polymeric materials. These clamps 1720 may have notches 1731 to lend some flexibility and areas of reduced material thickness to the otherwise fixed clamps. By way of the notches 1731, clamps 1720 may be configured to expand outward from a neutral position as the pusher 1711 is pressed to push collection pad out of the handpiece 1712. The overall configuration enhances the grip on the pad 1718, as well as participates in the release of the pad 1718 at the end of the collection process. As described further in FIGS. 18A-B, clamp 1720 at the distal end of the handpiece 1712, may hold the sample collection pad 1718 in the assembled position during shipment and fluid collection, and release the sample collection pad 1718, via the opposite movement of clamps 1720 away from each other, from the handpiece 1712 after fluid collection is completed.

Handpiece 1712 may be formed from first 1713a, and second 1713b body components. In some embodiments, the first 1713a and second 1713b body components are may be nonmirrored, mirrored, or complimentary, so details described for the first body component 1713a will be substantially the same for second body component 1713b. Body component 1713b has clamp 1720 terminal end. Clamp 1720 includes radially extending pad holder notched recesses 1731 that extend into clamp opening on either side of recess 1744, sized provide reduce thickness in the area of the notched recesses, enhance flexibility, and reduce force needed to expel collection pad 1718 from clamp. Although recess 1744 is illustrated as a full opening, in some embodiments, the recess 1744 may be an area of reduced thickness in the body component wall. Clamp 1720 may form a fixed opening that has a width greater than the collection pad with, in the range of 0.1 to 10 mm, for example 6 mm depending on thickness of the pad. Clamp 1720 may also include inner and outer guidewall ramps 1733, 1734 along a portion of the length of the arm 1713b to guide pusher 1711. Additional inner guidewall ramp 1733 may be formed at an end of the handpiece 1712 to serve as a stop and prevent further pushing of the knob end 1716 in a direction of the clamp 1720.

Clamp 1720 may include release ramps 1736 and pad retaining projects 1737, 1738. Pad retaining projections 1737 are small raised patterns such as spikes formed along the outer perimeter of each arm of the claim 1720 to provided additional frictional engagement with the pad 1718. Pad holder projections, such as pins and ribs, 1737 may extend towards each other on opposite side of collection pad 1718 to provide additional stability to the pad 1718 in the assembled position and prevent rotation of the pad. A pair of pad retaining projections 1738 may extend equally and oppositely towards each other from each arm of clamp 1720 to contact each other in the retaining testing position. In some embodiments, a pair of pad retaining projections 1738 may be formed on only a single one of the first or second body components 1713a,1713b so that the pad retaining projections 1738 extend fully through the collection pad in the retaining position. In some embodiments with pad retaining projections 1738 only on a single of the first or second body components 1713a, 1713b, the pad retaining projections 1738 may extend into a correspondingly shaped recess or other receiving component area on the opposite body component. In some embodiments pad retaining projections 1738 may utilize a spot adhesive on mating surfaces to bind the pad holding features to ensure the device does not eject the pad prior to use. Release ramps 1736 are configured to interact with corresponding ramped walls 1740 on inner shaft 1714 of pusher 1711 to cause force to open clamp 1720 by flexing at the pad holder notches 1731 and recess 1744, to separate the pad retaining projections 1738, and release the collection pad 1718. This may create a haptic feedback while overcoming the friction of the release ramps 1736. Additionally, release ramps 1736 may reduce unintended release of the pad due to accidental depression of the pusher. Release ramps 1736 may have an angle of incline from the longitudinal plane of the clamp surface of in a range of 15-45 degrees, and preferably 35 degrees.

Similar to pusher in FIG. 15, pusher 1711 may include a knob end 1716 with inner shaft 1714 formed as two longitudinally extending parallel arms 1748 with a channel or recess 1729 formed between the arms that is closed at one end by the knob end 1716 and open at the opposite end. Arms 1748 each include a ramped wall 1740 extending along its length on both sides of each arm 1748 and terminating prior to the distal end of each arm at a same position. Release ramps may have a sloped positive angle followed by a flat top so that the corresponding sloped surface of the ramped wall 1740 so that the force applied to the knob 1716 moves the sloped surfaces along each other until maximum opening of the clamp 1720 is reached at the flat parallel flat tops of the release ramps 1736 and ramped walls 1740. Beyond the terminal end of each ramped wall 1740, each arm 1748 has an enlarged flat push contact surface 1750 for contacting and pushing collection pad 1718 out of the handpiece 1712. In an assembled position, outer guidewall ramps 1734 are located outside of arm 1748 of pusher 1511, and inner guidewalls 1733 align with the interior side of the ramped wall 1540 of the pusher 1540, which also may align with the recess 1729. Guidewall ramps 1733, 1734 and stop cooperate with movement of the knob 1716 towards the ejector stop 1733 during motion to eject the sample collection pad 1718, allowing for single-handed operation of the collection device 1710. Pusher may travel a range of 1-50 mm to open the clamp, or 2-25 mm, or 2-3 mm. After the clamp, or jaw, has been opened by the pusher, the pusher may travel 1-100 mm, or 10 mm to eject or release the pad from the device 1710. The force required to depress the pusher may be in a range from 0.1-10 lbs, for example 2 lbs.

A sample volume adequacy indicator (SVAI) 1722 may include one or more sample volume adequacy indicator windows 1724, 1726, that align with each of the handpiece 1712 and as well as a SVAI wick 1728 that may signal a user visibly through the one or more SVAI windows 1724, when a sufficient volume of oral fluid has been collected. SVAI wick 1728 may be coupled in a recess 1729 in pusher 1711, for example by a friction or interference fit so that wick 1728 protrudes from the end of pusher 1711 in a direction towards a notched section of collection pad 1718.

Collection pad 1718 may include a notched section 1751 at one end sized and shaped to accommodate the end of the SVAI wick 1728 and two openings 1752 sized and shaped to receive projections 1738. Projections 1738 align with openings 1752 so that the projections 1738 extend through openings 1752 and contact a receiving feature or surface and assembled position.

Referring to FIG. 18A, an exemplary embodiment of an assembled oral fluid collection device 1810 is shown. The clamps 1820 are shown assembled and holding a sample collection pad 1818. The handpiece 1812 includes an opening through which knob portion 1816 of pusher extends out of handpiece 1812, with the SVAI windows 1824 positioned between the knob 1816 and clamp arms 1820. SVAI windows 1824 may be placed around the circumference of the handpiece 1812 so that the SVAI is viewable from all sides. In this embodiment, two SVAI windows 1824 are positioned equidistantly around the handpiece 1812. However, other numbers of SVAI windows and distances may be used. As illustrated in FIG. 18B, collection pad 1818 in the assembled position is secured via pad locking component via singled sided projections 1838 extending through pad opening.

According to FIG. 18C, collection device 1810 may further include radially extending fins 1806 extending a length along handpiece 1812 to prevent handpiece 1812 from extending further into a collection container. Fins enhance ability for single-handed ejection of collection pad by preventing the device from falling or completely entering into the sample tube or other collection container. Although this embodiment is illustrated on the handpiece of FIGS. 17-18, it may be used with other embodiments, for example, FIG. 15.

According to FIG. 18D, collection device 1810 may with a SVAI 1822, which may further include a unitary wick 1828, and collection pad 1818 arrangement in which the wick and collection pad are formed as a continuous structure. Continuous structure may be paddle shaped so that an enlarged end with a recess 1821 is formed in pusher 1811. Recess 1821 is formed and sized to receive a convex key 1819 in the handpiece. Additionally, teeth 1831 may be formed on top of ramp 1833 that extend toward and help retain wick 1828 in the assembled position and provide counter force to ejection of the collection pad. Notched or otherwise weakened areas 1817 may be formed at the junction of the pad 1818 and wick 1822. During ejection, a force applied by ends of pusher 1811 to the collection pad 1818 may act in opposition to the opposite end of wick 1828 being maintained in place on key 1819, and the optional teeth 1831 acting to retain the wick 1828, so that the wick and pad separate or tear at the weakened areas 1817 with minimal wick remaining on the collection pad to ensure consistent collection volume. Although this embodiment is illustrated on the handpiece of FIGS. 17-18, it may be used with other embodiments, for example, FIG. 15. As detailed above, the “tearing force” required is significantly less in the machine direction than the cross-direction of the fibers of the wick and pad. Therefore, the weakened areas may be parallel to the machine direction to provide a lower tear strength and cleaner tear than cutting.

FIGS. 19-22 further illustrate additional embodiments of disposable collection devices with varying collection pad release mechanisms. Devices may be made from thermoformed material or other manufacturing processes, molded as a single piece of material that is folded into final shape. For example, FIG. 19 illustrates a disposable collection device 1910 with a handpiece 1912 that may be formed of a thermoformed material and a collection pad 1918 that extends from the end of the handpiece 1912 with a proximal end of the collection pad having a thermoformed area of increased width 1901 to fit in the terminal end of in the handpiece 1912. Although described as thermoformed, other forms of manufacturing may be used. Pusher 1911 projects from an opposite end of handpiece 1912 in the assembled position with SVAI wick 1922 coupled to pusher 1911 and contacting collection pad 1918. Force may be applied to pusher 1911 to eject collection into a sample collection container. FIG. 20 illustrates disposable collection device 2010, in which handpiece 2012 may be made from cardboard or other recyclable material with pusher 2011 projecting from opposite end of handpiece 2012 to which collection pad 2018 is secured via a tab 2013 portion of the cardboard body of the handpiece 2012 folded to extend through an opening 2019 in the collection pad 2018. Force may be applied to pusher 2011 to eject collection pad from the tab of the handpiece into a collection container. FIG. 21 illustrates a disposable collection device 2110 with a SVAI 2122 and a deformable convex element 2103 protruding from a side of the handpiece 2112. Downward force on the convex element 2103 may permanently deform the element in an opposite, concave, manner so that a pre-tensioned elastic band 2104 that is stretched around the convex element and secured at the distal end of the handpiece 2112 is released and the force of the band 2104 pushes or releases the collection pad 2118 out of the handpiece 2112.

FIG. 22 illustrates a partial exploded view of a disposable collection device 2210 with a handpiece 2212 that may be formed of a thermoformed material and a collection pad 2218 that extends from the end of the handpiece 2212. Collection pad 2218 may include two loading prongs 2219. Handpiece 2212 includes a receiver 2211, a recess 2209, and an ejection button 2208. An elastomeric band, such as a rubber band may be mounted to the handpiece in tension around the perimeter of the handpiece 2212. In order to assemble the collection device 2210, prongs 2219 may be pressed against the rubber band that is in tension across recess 2209 so that the prongs 2219 extend into and couple with a correspondingly sized and shaped projection in the interior of the handpiece 2212 so that the rubber band is further tensioned against the prongs 2219 inside the handpiece 2212. By pressing down on button 2208, interior projections coupled to button 2208 may move in a direction away from contacting prongs 2219, for example like a cantilever mechanism, so that the pre-tensioned rubber band ejects the collection pad 2218 from the handpiece 2212.

In some embodiments, SVAI windows may be positioned along handpiece at a location sufficiently distal from the collection pad so that both a test user and a test administrator can view that sufficient fluid has been collected by viewing the window without removing the pad from the test user's mouth. In some embodiments, handpiece and other component are made of a clear material such as transparent plastic so that the users can watch the SVAI indicator travel from the starting position to the SVAI windows. In some embodiments, the SVAI windows may form a continuous window around the entire circumference of the handpiece to be viewed at any angle.

Disclosed embodiments have described devices, kits, and methods for collecting and establishing sufficiency of fluid collection for testing in terms of oral fluid for humans or animals. However, as noted hereinabove, it is also envisioned that the devices, kits, and methods could be used for other types of fluid collection in humans or animals as well as in other environments where sufficiency of collection is important.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. An oral fluid collection device comprising:
a handpiece,
a sample volume adequacy indicator (SVAI) integrated into the handpiece and configured to provide a visual indicator of sufficient fluid collection, and
means for retaining a collection pad so that the collection pad is detachably coupled to the handpiece during oral fluid collection and ejected from the handpiece after oral fluid collection so that only the collection pad is coupled and decoupled from the handpiece.

2. The oral fluid collection device of claim 1, wherein the means for retaining comprises a clamp configured to retain a portion of the collection pad within a distal end of the handpiece when the collection pad is coupled to the handpiece.

3. The oral fluid collection device of claim 1, further comprising an inner shaft, wherein a joint is integrally formed with or coupled to the inner shaft, and wherein longitudinal movement of the inner shaft towards the distal end of the handpiece ejects the collection pad from the handpiece and the clamp.

4. The oral fluid collection device of claim 1, wherein the clamp comprises two opposing arms with substantially smooth surfaces, with release ramps and a pad locking component, or with release ramps and pad retaining projections.

5. The oral fluid collection device of claim 1, wherein the clamp comprises two opposing arms each having a plurality of perforations or surface texture configured to couple and decouple the collection pad.

6. The oral fluid collection device of claim 1, wherein the clamp is formed from one perforated continuous film, that folds around one end of the collection pad and is prevented from being ejected along with the pad by attachment to the inner shaft.

7. The oral fluid collection device of claim 1, further comprising an inner shaft, wherein the SVAI comprises windows in the handpiece, and a wick is positioned in the inner shaft so that a portion of the wick is visible through the aligned handpiece windows.

8. The oral fluid collection device of claim 1, wherein the wick is positioned in contact with the collection pad when the collection pad is coupled to the handpiece, and wherein the wick further comprises a visual indicator configured to migrate via a wicking action with the application of saliva to the aligned handpiece windows.

9. The oral fluid collection device of claim 1, wherein one or more of the inner shaft and handpiece are constructed so to prevent relative rotation of the inner shaft with respect to the handpiece.

10. The oral fluid collection device of claim 1, wherein the wick and the collection pad are one piece.

11. An oral fluid collection kit comprising:
an oral fluid collection device having a handpiece, a sample volume adequacy indicator (SVAI) integrated into the handpiece and configured to provide a visual indicator of sufficient fluid collection, and a clamp configured to retain a portion of the collection pad within a distal end of the handpiece when the collection pad is detachably coupled to the handpiece and to eject only the collection pad from the handpiece, and
a collection container configured to receive the collection pad when ejected from the handpiece, wherein the collection container contains a buffer solution.

12. The oral fluid collection kit of claim 11, wherein the buffer comprises a buffer containing a detergent and preservative.

13. The oral fluid collection kit of claim 11, wherein the buffer is a neutral phosphate buffer.

14. The oral fluid collection kit of claim 11, further comprising an inner shaft, wherein the clamp is integrally formed with or coupled to the inner shaft, and wherein longitudinal movement of the inner shaft towards the distal end of the handpiece ejects the collection pad from the handpiece and the dual lever clamp, or wherein the clamp is integrally formed with the handpiece and the collection pad is coupled via the clamp, wherein longitudinal movement of the inner shaft towards the distal end of the handpiece ejects the collection pad from the handpiece and the clamp.

15. The oral fluid collection kit of claim 11, further comprising an inner shaft, wherein the SVAI comprises windows in the handpiece that are aligned with windows in the inner shaft, and a wick positioned in the inner shaft so that a portion of the wick is visible through the aligned handpiece.

16. The oral fluid collection kit of claim 11, wherein the wick is positioned in contact with the collection pad when the collection pad is coupled to the handpiece, and wherein the wick further comprises a visual indicator configured to migrate via a wicking action with the application of saliva to the aligned handpiece and inner shaft windows.

17. The oral fluid collection kit of claim 11, wherein the wick and the collection pad are one piece.

18. A method of manufacturing an oral fluid collection device comprising:
forming a handpiece and at least one inner shaft component, and a knob,
inserting a wick into the at least one inner shaft component, and inserting the wick with the at least one inner shaft component into the handpiece to form an sample volume adequacy indicator (SVAI); and
coupling a collection pad to the at least one inner shaft component or the handpiece so that the collection pad contacts the wick.

19. The method of claim 18, wherein the at least one inner shaft component comprises two inner shaft components formed to include a dual lever joint that is integrally formed with or coupled to the inner shaft, and the collection pad is coupled via the dual lever joint, or wherein a clamp is integrally formed with the handpiece and the collection pad is coupled via the clamp.

20. The method of claim 18, wherein the SVAI comprises windows in the handpiece, and the wick is positioned in the inner shaft so that a portion of the wick is visible through the aligned handpiece windows.
